SEQUENCE LISTING (64 sequences)

SEQ ID NO: 1 - DNA, 365 bp, Homo sapiens
  CDS (298)...(365)
ggaggataggaagcaggaaagcgggagagctcgagggacaagggggctcggtgtgtttacaccaggcacgggctacgagcgtccatcccggcccctggcttgcgctcccgaagaggagagcaaggctgttctgggatccggccgtcgtgcggcaagaggcttgtctgtccgggttgccggaaccaggagaacccagagggaaaccgagggaaaggagcggcgcgttttactagagagagcgcgagcggaagaggcgagagcaggagcgcgcgagggagcatcgagcgcagcggagacatgaggacctactggctgcacagcgtctgggtgctgggctttttcctgtccctcttctcattgcaagg

SEQ ID NO: 2 - protein, 22 aa, Homo sapiens
MRTYWLHSVWVLGFFLSLFSLQ

SEQ ID NO: 3 - DNA, 365 bp, Homo sapiens
ccttgcaatgagaagagggacaggaaaaagcccagcacccagacgctgtgcagccagtaggtcctcatgtctccgctgcgctcgatgctccctcgcgcgctcctgctctcgcctcttccgctcgcgctctctctagtaaaacgcgccgctcctttccctcggtttccctctgggttctcctggttccggcaacccggacagacaagcctcttgccgcacgacggccggatcccagaacagccttgctctcctcttcgggagcgcaagccaggggccgggatggacgctcgtagcccgtgcctggtgtaaacacaccgagcccccttgtccctcgagctctcccgctttcctgcttcctatcctcc

SEQ ID NO: 4 - DNA, 655 bp, Homo sapiens
ctgaggatggctgtgtccccctgcctcacggtgatgttgtccgtgcctcggttaaaatccacgctgcgaacaggcagtcctgtgggaagaaggcagagcaatctcagtaggaccagtggcaactgtttccgatccggctgaactctcctgaccatggtggccacgccgaggtgcgggtccgcggggtgctctggaggggtgcgcgctgctcgcgaggagaggcttcaccaacacggggctttcatccacagcgagcgcagagcgggctttgccagtttatggtcctttccactttgcctctctctttccctcgctcagtctcttttccctctaagacttaacaaagccctcataaaacccaagcagaagggtgaaaagtaaaagcaacacaatttcaaaagagagagtgcaaatagcaagccctctggaagagctgaagaggaagccaaaggaaagggttcttgtttggtctctctgtgactggatgctcctctgccagtgtctgagctgagctccttcgctctgtaacccactttcccaggctggcgggcgggcgggcgagggagccggcaccaagcctgccagtgagtgtacagaaacagccacacagcagcagcagcagcagaagcagcagcaacccagagcctctctcccc

SEQ ID NO: 5 - protein, 25 aa, Homo sapiens
MVRRVQPDRKQLPLVLLRLLCLLPT

SEQ ID NO: 6 - DNA, 1640 bp, Homo sapiens
ggggagagaggctctgggttgctgctgcttctgctgctgctgctgctgtgtggctgtttctgtacactcactggcaggcttggtgccggctccctcgcccgcccgcccgccagcctgggaaagtgggttacagagcgaaggagctcagctcagacactggcagaggagcatccagtcacagagagaccaaacaagaaccctttcctttggcttcctcttcagctcttccagagggcttgctatttgcactctctcttttgaaattgtgttgcttttacttttcacccttctgcttgggttttatgagggctttgttaagtcttagagggaaaagagactgagcgagggaaagagagaggcaaagtggaaaggaccataaactggcaaagcccgctctgcgctcgctgtggatgaaagccccgtgttggtgaagcctctcctcgcgagcagcgcgcacccctccagagcaccccgcggacccgcacctcggcgtggccaccatggtcaggagagttcagccggatcggaaacagttgccactggtcctactgagattgctctgccttcttcccacaggactgcctgttcgcagcgtggattttaaccgaggcacggacaacatcaccgtgaggcagggggacacagccatcctcaggtgcgttgtagaagacaagaactcaaaggtggcctggttgaaccgttctggcatcatttttgctggacatgacaagtggtctctggacccacgggttgagctggagaaacgccattctctggaatacagcctccgaatccagaaggtggatgtctatgatgagggttcctacacttgctcagttcagacacagcatgagcccaagacctcccaagtttacttgatcgtacaagtcccaccaaagatctccaatatctcctcggatgtcactgtgaatgagggcagcaacgtgactctggtctgcatggccaatggccgtcctgaacctgttatcacctggagacaccttacaccaactggaagggaatttgaaggagaagaagaatatctggagatccttggcatcaccagggagcagtcaggcaaatatgagtgcaaagctgccaacgaggtctcctcggcggatgtcaaacaagtcaaggtcactgtgaactatcctcccactatcacagaatccaagagcaatgaagccaccacaggacgacaagcttcactcaaatgtgaggcctcggcagtgcctgcacctgactttgagtggtaccgggatgacactaggataaatagtgccaatggccttgagattaagagcacggagggccagtcttccctgacggtgaccaacgtcactgaggagcactacggcaactacacctgtgtggctgccaacaagctgggggtcaccaatgccagcctagtccttttcagacctgggtcggtgagaggaataaatggatccatcagtctggccgtaccactgtggctgctggcagcatctctgctctgccttctcagcaaatgttaatagaataaaaatttaaaaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - protein, 338 aa, Homo sapiens
MVRRVQPDRKQLPLVLLRLLCLLPTGLPVRSVDFNRGTDNITVRQGDTAILRCVVEDKNSKVAWLNRSGIIFAGHDKWSLDPRVELEKRHSLEYSLRIQKVDVYDEGSYTCSVQTQHEPKTSQVYLIVQVPPKISNISSDVTVNEGSNVTLVCMANGRPEPVITWRHLTPTGREFEGEEEYLEILGITREQSGKYECKAANEVSSADVKQVKVTVNYPPTITESKSNEATTGRQASLKCEASAVPAPDFEWYRDDTRINSANGLEIKSTEGQSSLTVTNVTEEHYGNYTCVAANKLGVTNASLVLFRPGSVRGINGSISLAVPLWLLAASLLCLLSKC

SEQ ID NO: 8 - protein, 335 aa, Homo sapiens
MRTYWLHSVWVLGFFLSLFSLQGLPVRSVDFNRGTDNITVRQGDTAILRCVVEDKNSKVAWLNRSGIIFAGHDKWSLDPRVELEKRHSLEYSLRIQKVDVYDEGSYTCSVQTQHEPKTSQVYLIVQVPPKISNISSDVTVNEGSNVTLVCMANGRPEPVITWRHLTPTGREFEGEEEYLEILGITREQSGKYECKAANEVSSADVKQVKVTVNYPPTITESKSNEATTGRQASLKCEASAVPAPDFEWYRDDTRINSANGLEIKSTEGQSSLTVTNVTEEHYGNYTCVAANKLGVTNASLVLFRPGSVRGINGSISLAVPLWLLAASLLCLLSKC

SEQ ID NO: 9 - DNA, 1283 bp, Homo sapiens
ctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 10 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 11 - DNA, 200 bp, Homo sapiens
atgaaactttctctaaactattagtagtgcttttccaaatggataaaagcacattttgcagaataatgtaatataattattttctcaacattttgccttaattataatcagtttcataatttaaagcattcatagtttgagaaatgtaagccaaagaataatgcatttgttttctaaattattatcccctgattgagtta

SEQ ID NO: 12 - DNA, 326 bp, Homo sapiens
tagccttgtagataaactgcaatagcataaaaataacaaaatttgcagctgccaaaatttatcctacctttgacatttatggactggtgggtgtgagaaagttatcaaatccctaggaaattcagtttcctctttaaaaaaaaatggggaccatgatacctaacttgcaagaaaatagatgtagcacactacaatgtgagccacaatgctttatttattcaaaactctaaaattccagcaaactgagggaaagtgtgaaaggttcatggggctggccgtaagagccttcgtgtacctcttggttcctctgtgccagcaaacagaag

SEQ ID NO: 13 - DNA, 42 bp, Homo sapiens
tctctttctgtctcagacagacatattaaaatgaactagatt

SEQ ID NO: 14 - DNA, 435 bp, Homo sapiens
agtaatacctaatgagcacccttaagtgtaaaataatagttgctttaaaataatttttaaaaaataaactatattgtcaacattaaattaagggcagaaaaatgtattaaagttctaaaactacagaatgaataattgaaatattctaacctagaagagtaaaaaattggtgaatgtcattaaaggtttctaacagaagatccactaggaagagaagtgggatacaaattgctagtaagaaaagggagagggggaatagaatataccgtatgtttaaaacttccaacatccttttcctagcccctgatctatgcaaaatgaagtcttactaagtctcaaaacagtattctttacatttcattttctttctttttcaatcattgtttttgagaaaatcctaaaccaaagcaaaaaagagagaatctgccagtgaag

SEQ ID NO: 15 - DNA, 51 bp, Homo sapiens
aaattattatcccctgattgagttartagccttgtagataaactgcaatag

SEQ ID NO: 16 - DNA, 51 bp, Homo sapiens
cagacatattaaaatgaactagattragtaatacctaatgagcacccttaa

SEQ ID NO: 17 - DNA, 21 bp, Homo sapiens
ttgactgacgttcttccatga

SEQ ID NO: 18 - DNA, 20 bp, Homo sapiens
agggaaagggcatatggagt

SEQ ID NO: 19 - DNA, 20 bp, Homo sapiens
taaggttttcgaggggaggt

SEQ ID NO: 20 - DNA, 20 bp, Homo sapiens
atcagcgaacctgctcaaag

SEQ ID NO: 21 - DNA, 20 bp, Homo sapiens
tgtttctcagccagggttgt

SEQ ID NO: 22 - DNA, 20 bp, Homo sapiens
tggttgatcaatgcaaatcc

SEQ ID NO: 23 - DNA, 21 bp, Homo sapiens
ttgagagtgaaggggtttgag

SEQ ID NO: 24 - DNA, 20 bp, Homo sapiens
tttcccttccaaaacattgc

SEQ ID NO: 25 - DNA, 21 bp, Homo sapiens
tgttcacagtgggagtgttgg

SEQ ID NO: 26 - DNA, 24 bp, Homo sapiens
ggcatgaaacatatttgaggctta

SEQ ID NO: 27 - DNA, 22 bp, Homo sapiens
agccaggctaactgtgttcaag

SEQ ID NO: 28 - DNA, 22 bp, Homo sapiens
ggcctaagatggggaatgaaat

SEQ ID NO: 29 - DNA, 23 bp, Homo sapiens
ttgccttactttacctcttctgc

SEQ ID NO: 30 - DNA, 23 bp, Homo sapiens
ttctgcccttaatttaatgttga

SEQ ID NO: 31 - DNA, 21 bp, Homo sapiens
tgcggtaatcactatcccaag

SEQ ID NO: 32 - DNA, 21 bp, Homo sapiens
catcttgaagtgaccctggag

SEQ ID NO: 33 - DNA, 20 bp, Homo sapiens
ccgaaagaggaaatgctttg

SEQ ID NO: 34 - DNA, 20 bp, Homo sapiens
acgacaacccctaccatttg

SEQ ID NO: 35 - DNA, 20 bp, Homo sapiens
ggcttggtcatggaaattgt

SEQ ID NO: 36 - DNA, 20 bp, Homo sapiens
atcctcccaaccccttactc

SEQ ID NO: 37 - DNA, 20 bp, Homo sapiens
ccgctggcttgtgaataact

SEQ ID NO: 38 - DNA, 20 bp, Homo sapiens
cctgaaactgggggtagtca

SEQ ID NO: 39 - DNA, 21 bp, Homo sapiens
ccccatgtcatttctactcca

SEQ ID NO: 40 - DNA, 20 bp, Homo sapiens
gtggcatctttgggatcaat

SEQ ID NO: 41 - DNA, 24 bp, Homo sapiens
cccagaatttttagagaaatcgaa

SEQ ID NO: 42 - DNA, 23 bp, Homo sapiens
tcctctgctgttatcttttcagc

SEQ ID NO: 43 - DNA, 20 bp, Homo sapiens
tgggaaagctttttggattg

SEQ ID NO: 44 - DNA, 20 bp, Homo sapiens
cgtggttctcaggtaggaca

SEQ ID NO: 45 - DNA, 20 bp, Homo sapiens
acagtacacaggcacccaca

SEQ ID NO: 46 - DNA, 26 bp, Homo sapiens
tttttctgtgatttgagattgttctt

SEQ ID NO: 47 - DNA, 20 bp, Homo sapiens
ttttatggccaaagccagtc

SEQ ID NO: 48 - DNA, 20 bp, Homo sapiens
ggggtgtttgggtaagaatg

SEQ ID NO: 49 - DNA, 25 bp, Homo sapiens
gacttgtgcaagttaaaacttgaaa

SEQ ID NO: 50 - DNA, 22 bp, Homo sapiens
gagtagcctggggatgagcaaa

SEQ ID NO: 51 - DNA, 26 bp, Homo sapiens
ctttgaatcccatgatgatagattga

SEQ ID NO: 52 - DNA, 22 bp, Homo sapiens
aggaaagccgtctgaggaggag

SEQ ID NO: 53 - DNA, 27 bp, Homo sapiens
ctttagaattgtaatggtcatctcgac

SEQ ID NO: 54 - DNA, 24 bp, Homo sapiens
cacttgagtaaatgggcagaagat

SEQ ID NO: 55 - DNA, 27 bp, Homo sapiens
cacttaagggtgctcattaggtattac

SEQ ID NO: 56 - DNA, 20 bp, Homo sapiens
aggaaacagtgcattggggc

SEQ ID NO: 57 - DNA, 18 bp, Homo sapiens
ggcgggaccgcgagttaa

SEQ ID NO: 58 - DNA, 25 bp, Homo sapiens
gcagcttcatcagatcaaggacatt

SEQ ID NO: 59 - DNA, 23 bp, Homo sapiens
gtggctccctacagttggggttc

SEQ ID NO: 60 - DNA, 27 bp, Homo sapiens
ggaaacttttggaatctcctattcatc

SEQ ID NO: 61 - DNA, 26 bp, Homo sapiens
tatcttttcagctaatgcatcttcca

SEQ ID NO: 62 - DNA, 23 bp, Homo sapiens
gaagataggaacagtcacatagc

SEQ ID NO: 63 - DNA, 24 bp, Homo sapiens
gtgttccagggcattaattgtgtc

SEQ ID NO: 64 - DNA, 24 bp, Homo sapiens
ccatgacaggaatgtggatataca

(19) United States
(12) Patent Application Publication
(10) Pub. No.: US 2011/0070583 A1
(43) Pub. Date: Mar. 24, 2011

(54) Lsamp gene associated with cardiovascular disease

(75) Inventors: Jeffery M. Vance, Chapel Hill, NC (US); Pascal Goldschmidt, Miami, FL (US); Elizabeth Hauser, Durham, NC (US); William Kraus, Hillsborough, NC (US); Margaret A. Pericak-Vance, Chapel Hill, NC (US)

(73) Assignee: Duke University

(21) Appl. No.: 12/806,690

(22) Filed: Aug. 19, 2010

Related U.S. Application Data
(62) Division of application No. 11/458,228, filed on Jul. 18, 2006, now abandoned.
(60) Provisional application No. 60/709,800, filed on Aug. 22, 2005.
(60) Provisional application No. 60/700,301, filed on Jul. 19, 2005.

(51) Int. Cl. C12Q 1/68; C12Q 1/02; C12Q 1/60

(52) U.S. Cl. 435/6; 435/29; 435/11

(57) ABSTRACT

The LSAMP gene can be used for cardiovascular disease risk assessment, in particular Left Main Disease. The genetic risk attributable to LSAMP adds to known cardiovascular disease risk factors. Assessment of risk attributable to LSAMP permits early initiation of preventive and therapeutic strategies. Given the pronounced clinical risk associated with Left Main Disease, such risk assessment should significantly reduce morbidity and mortality.

### STATEMENT OF PRIORITY

This application is a divisional application of, and claims priority to, U.S. Application Ser. No. 11/458,228, filed Jul. 18, 2006 (pending), which claims the benefit, under 35 U.S.C. §119(e), of U.S. Provisional Application Ser. No. 60/709,800, filed Aug. 22, 2005 and U.S. Provisional Application Ser. No. 60/700,301, filed Jul. 19, 2005, the entire contents of each of which are incorporated by reference herein.

This invention was made using funds from the United States Government under Grant No. P01HL73042 from the National Institutes of Health. The government therefore retains certain rights in the invention according to the terms of the grant.

### TECHNICAL FIELD OF THE INVENTION

This invention is related to the area of risk assessment and drug discovery. In particular, it relates to assessment and drugs for treating cardiovascular disease.

### BACKGROUND OF THE INVENTION

Coronary artery disease (CAD) is a leading cause of death and disability in modern society. Epidemiological studies have repeatedly shown that a positive family history is a robust predictor of CAD, even after adjustment for all known risk factors, suggesting the existence of a substantial genetic component for CAD (1; 2). To date, five genomic linkage scans for CAD have been conducted (3-7). A meta-analysis of four of these studies confirmed a susceptibility locus on chromosome 3q26-27 (8). However, the gene or genes contributing to CAD risk in this region have yet to be identified. Most recently, we reported one of the largest genome scans for early-onset CAD, the GENECARD study (9). The most significant evidence for linkage was found at chromosome 3q13 (multipoint LOD score=3.5; OMIM: 608901), with a peak near the microsatellite marker D3S2460. We present here association studies in an independent case-control dataset (CATHGEN) to identify the gene contributing to the chromosome 3q13 CAD locus.

There is a continuing need in the art to identify factors contributing to cardiovascular disease and to identify drugs for treating cardiovascular disease.

### SUMMARY OF THE INVENTION

One embodiment of the invention provides a method to aid in predicting risk of cardiovascular disease. Expression level of exon 1a of LSAMP in a human cardiovascular tissue sample is determined. The determined expression level of exon 1a of LSAMP is compared to expression data from a population of control humans. Risk of cardiovascular disease is predicted based on the determined expression level.

Another embodiment of the invention is a method to aid in predicting risk of cardiovascular disease. Presence in a human's genome of a G allele of SNP rs1875518 or an A allele of rs1676232 is determined. The human is identified as having a high risk of cardiovascular disease if the human has said G allele or said A allele.

Yet another embodiment of the invention is a method of screening compounds to identify candidate drugs for preventing cardiovascular disease. A cell is contacted with a test compound. Expression level of exon 1a of LSAMP in the cell is determined. A test compound is identified as a candidate drug for preventing cardiovascular disease if it increases expression of exon 1a of LSAMP.

Still another aspect of the invention is a method of screening compounds to identify candidate drugs for preventing cardiovascular disease. A nucleic acid comprising a human LSAMP gene is contacted in vitro with a test compound and with reagents for transcription of said human LSAMP gene. Transcription level of exon 1a of LSAMP is determined. A test compound is identified as a candidate drug for preventing cardiovascular disease if it increases expression of exon 1a of LSAMP.

Another aspect of the invention is a method for detecting the presence in an individual of an allele which predisposes humans to develop cardiovascular disease. The presence or absence of a DNA polymorphism on human chromosome band 3q13.32 in a DNA sample isolated from an individual is determined. The presence of said DNA polymorphism is correlated with the presence of cardiovascular disease.

Yet another aspect of the invention is a method for detecting the presence in an individual of an allele which predisposes an individual to develop cardiovascular disease. A polymorphism on human chromosome band 3q13.32 which is linked to Left Main Coronary Artery Disease phenotype in a set of affected familial relatives of an individual is determined. The individual is tested for the presence of said polymorphism. The presence of the polymorphism in the individual indicates that the individual is at high risk of Left Main Coronary Artery Disease.

Another embodiment of the invention provides an isolated antibody composition which specifically binds to a human LSAMP protein comprising a sequence as shown in SEQ ID NO: 2 (exon 1a), but which does not bind to a human LSAMP protein comprising a sequence as shown in SEQ ID NO: 5 (exon 1b).

According to another aspect of the invention a kit is provided to aid in predicting risk of cardiovascular disease. The kit comprises one or more components in a divided or undivided container. One such component is an antibody which specifically binds to an LSAMP protein comprising a sequence as shown in SEQ ID NO: 2 (exon 1a) but which does not bind to a protein comprising a sequence as shown in SEQ ID NO: 5 (exon 1b).

Another embodiment of the invention is a kit to aid in predicting risk of cardiovascular disease. The kit comprises one or more components in a divided or undivided container. One such component is a pair of primers for amplifying a single nucleotide polymorphism (SNP) marker selected from the group consisting of rs1676232 and rs1875518. Another component is a probe that hybridizes to the SNP marker and which includes an A or G at the single polymorphic nucleotide or which has its 3′ terminus immediately adjacent to the single polymorphic nucleotide.

Still another embodiment of the invention is yet another kit to aid in predicting risk of cardiovascular disease. The kit comprises one or more components in a divided or undivided container. Two such components are a forward and a reverse primer for amplifying a human LSAMP cDNA. The cDNA comprises exon 1a. Each primer comprises at least 12 nucleotides selected from contiguous nucleotides of SEQ ID NO: 1 and 3, respectively.

A further embodiment of the invention is a cDNA molecule which encodes an LSAMP protein according to SEQ ID NO: 8 or which is at least 95% identical to a cDNA molecule comprising nt 298-365 of SEQ ID NO: 1 and nt 576-1517 of SEQ ID NO: 6. The LSAMP protein is encoded by a transcript which includes exon 1a.

Yet a further embodiment of the invention is an oligonucleotide comprising at least 18 contiguous nucleotides of exon 1a of LSAMP according to SEQ ID NO: 1. The oligonucleotide can be used, inter alia, to quantitate expression of a transcript comprising exon 1a.

According to another aspect of the invention, an isolated and purified LSAMP protein is provided. The protein comprises an amino acid sequence according to SEQ ID NO: 8 or is at least 95% identical to SEQ ID NO: 8.

Another aspect of the invention provides one or more computer readable media storing computer executable instructions which when executed by a data processing device perform a method. Input data corresponding to a determined expression level of exon 1a of LSAMP in a human is received. The input data is compared to expression data of expression level of exon 1a of LSAMP from a population of control humans. A risk value corresponding to a risk of cardiovascular disease in the human is determined based on the comparison.

Another aspect of the invention provides one or more computer readable media storing computer executable instructions which when executed by a data processing device perform a method. Input data corresponding to genomic DNA of a human is received. The input data is analyzed to determine presence in the human's genome of an allele of SNP rs1875518 or an allele of SNP rs1676232. A risk value is determined corresponding to a human's risk of cardiovascular disease based on the allele of the SNP determined.

Still another aspect of the invention provides one or more computer readable media storing computer executable instructions which when executed by a data processing device perform a method. Input data corresponding to DNA of a human is received. The input data is analyzed to determine presence or absence of a DNA polymorphism on human chromosome band 3q13.32 in the human. The presence or absence of said DNA polymorphism is correlated with the presence of cardiovascular disease. A risk value corresponding to the human's risk of cardiovascular disease is determined based on presence or absence of the DNA polymorphism.

Yet another aspect of the invention provides one or more computer readable media storing computer executable instructions which when executed by a data processing device perform a method. Input data corresponding to DNA of a human is received. The input data is analyzed to determine presence or absence in the human of a polymorphism on human chromosome band 3q13.32 which is linked to Left Main Coronary Artery Disease phenotype in a set of affected familial relatives of the human. A risk value corresponding to the human's risk of Left Main Coronary Artery Disease is determined.

Still another aspect of the invention provides one or more computer readable media having stored thereon a data structure. The structure comprises data fields. A first data field contains data identifying a patient. A second data field contains data corresponding to the patient. The data corresponding to the patient is selected from the group consisting of: expression level of exon 1a of LSAMP; an allele of SNP rs1875518; an allele of SNP rs1676232; a DNA polymorphism on human chromosome band 3q13.32 correlated with the presence of cardiovascular disease; and a DNA polymorphism on human chromosome band 3q13.32 which polymorphism is linked to Left Main Coronary Artery Disease phenotype in a set of affected familial relatives of the patient. A third data field contains data corresponding to the patient selected from the group consisting of level of triglycerides, levels of cholesterol, diabetes mellitus, hypertension, family history, cigarette smoking, echocardiogram results, stress test results, blood pressure measurement, and an ejection fraction measure.

These and other embodiments which will be apparent to those of skill in the art upon reading the specification provide the art with reagents and methods for detection, diagnosis and drug screening pertaining to cardiovascular disease.

### BRIEF DESCRIPTION OF THE DRAWINGS

FIGS. 1A-1C. Overview of fine mapping on Chromosome 3. FIG. 1A. Major susceptibility loci for CAD were mapped to chromosome 3q13 in the GENECARD study (9). A multipoint nonparametric LOD score curve on chromosome 3 is displayed in a customized Ensembl genome browser tract (37). The red box indicates the chromosomal region surveyed in the initial DNA pooling screen. FIG. 1B. The physical locations of the 16 initial screening SNPs are displayed. The marker density is approximately one SNP every 150 Kb. The peak microsatellite marker D3S2460 (GENECARD) is displayed as a thick bar. The significant pooling SNP rs1875518 is underlined. FIG. 1C. Physical locations of the 36 SNPs examined in the high-density follow-up stage. SNP rs1875518 is underlined for reference. (CAGACATATTAAAATGAACTAGATT [A/G] AGT AATA CCTAATGAGCACCCTTAA; SEQ ID NO: 16) The most significant SNP, rs1676232, is circled. (AAATTATTATCCCCTGATTGAGTTA [A/G] TAGCCTTGT AGATAAACTGCAATAG (SEQ ID NO: 15)

FIG. 2. Initial association analysis on SNPs surrounding rs1875518 in different case groups. Each point represents an additive association test adjusted for gender and ethnicity on one SNP between the control dataset (N=204) and the different case datasets: young affected (YA_aac, N=301, circle), old affected (OA_aac, N=168, triangle), or GENECARD probands (GC, N=420, square). The significant marker originally identified by DNA pooling (rs1875518), the most significant marker identified by individual genotyping (rs1676232), the only significant marker associated with YA (rs2937666), and other markers that were significant in both GENECARD probands and CATHGEN old affected (rs 1354152, rs 1698041, rs2055426, and rs2937675) are labeled. Additional analyses on SNPs lying in between the two vertical bars are reported in Table 2.

FIGS. 3A-3B. FIG. 3a. LSAMP_{—}1a expression is downregulated in aortas with severe atherosclerosis burden. FIG. 3b. Lower expression level of LSAMP_{—}1a is associated with the risk genotype of rs1676232. Total RNAs were extracted from 37 human aortas (15). The expression of the LSAMP_{—}1a was measured by TaqMan® real-time RT-PCR in triplicates. The mean of the multiple measurements of each aorta was used to calculate the mean of each category. Each bar represents the mean±SEM from all the samples in one category.

FIG. 4. (Supplementary FIG. 1). Allele frequency difference estimated by allelotyping of DNA pools. Each bar represents the allele frequency difference between two groups, as estimated by DNA pooling with three replicates in each group. The bar with * Indicates a significant allele frequency difference between two groups (z-test). White bar, YA_aac (CADi >23, age-at-catheterization <56, N=301) versus control (N=204); gray bar, OA_aac (CADi >67, age-at-catheterization 56, N=168) versus control.

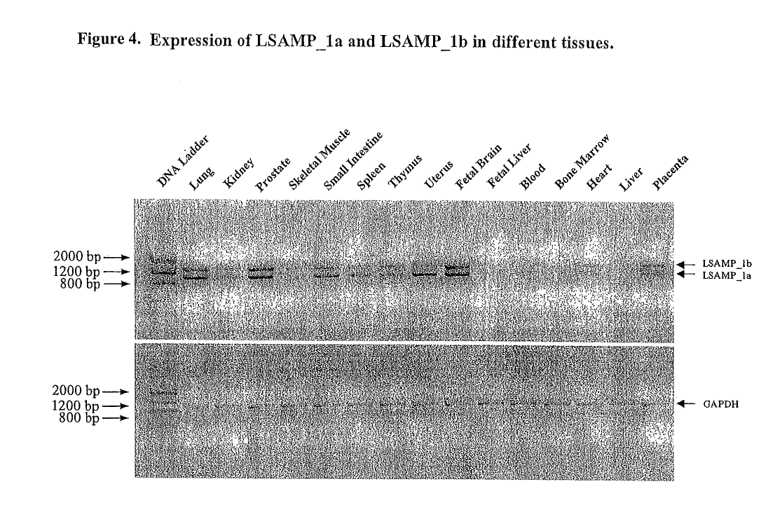

FIG. 5. (Supplementary FIG. 2.) Expression of LSAMP_{—}1a and LSAMP_{—}1b in different tissues. Total RNA from different human tissues was purchased from BD biosciences (Human Total RNA Master Panel II). RT-PCR was used to examine the expression of LSAMP_{—}1a and LSAMP_{—}1b in each tissue. RT-PCR products of GAPD from each tissue were displayed in the lower panel. Low DNA Mass Ladder from Invitrogen was used to indicate molecular weight.

FIG. 6. (Supplementary FIG. 3.) Expression of LSAMP_{—}1a and LSAMP_{—}1b in human aortic endothelial cells and smooth muscle cells. Total RNAs were isolated from normal human aortic endothelial cells and smooth muscle cells (Cabrex Bio Science). RT-PCR was used to examine the expression of LSAMP_{—}1a and LSAMP_{—}1b in each type of cell. RT-PCR products of GAPD from each type of cell were displayed in the lower panel. Low DNA Mass Ladder from Invitrogen was used to indicate molecular weight.

FIG. 7. (Supplementary Table 3.) Pairwise linkage disequilibrium analysis between SNPs in control subjects. Pairwise LD between 29 SNPs with MAF greater than 2% was estimated in the controls; top-right triangle details the square of the correlation coefficient (r²) and the bottom-left triangle details the standard disequilibrium coefficient (D′). A similar pattern of LD was observed in the affected dataset and is not reported. Values >0.9 are in shaded grey.

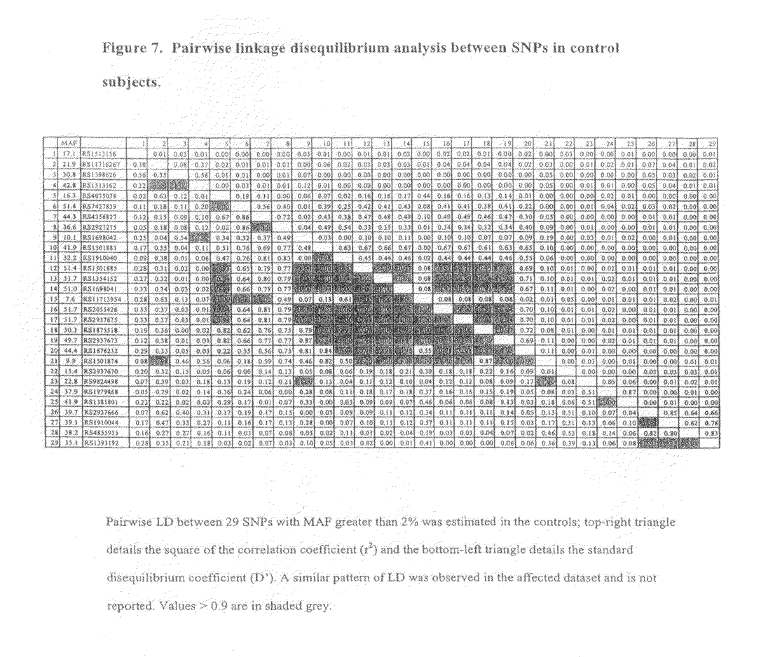

### DETAILED DESCRIPTION OF THE INVENTION

We describe a susceptibility locus within the LSAMP gene that is strongly associated with cardiovascular disease, in particular with LMD. This association was found in two independent case datasets (GENECARD and CATHGEN) as well as in a dataset from a recent study reporting a high heritability for CAD involving the left main coronary artery but not for more peripheral coronary lesions (19). Our data indicate that LSAMP is a cardiovascular disease risk gene: it is down-regulated in aortas with severe atherosclerosis; and lower expression of the gene is coupled with the risk allele of the most significant SNP marker in LSAMP gene in the third independent dataset.

Cardiovascular diseases for which the present invention can be used include, without limitation, coronary artery disease, arteriosclerosis, and left main disease. Samples for genetic testing can be taken from any tissue in the body that is convenient, including but not limited to blood cells, skin cells, cheek cells. Samples for testing expression are preferably taken from a cardiovascular tissue, including coronary artery and aorta. More preferably the sample is taken from smooth muscle cells of the cardiovascular tissue. Surgically removed tissue can be tested, such as that from a biopsy.

For testing expression of LSAMP, either mRNA or protein can be determined. Any method known in the art for determining and quantifying mRNA or protein can be used. Many such methods are known and can be used as is convenient. These include without limitation, RT-PCR, Western blots, Northern blots, ELISA, immunoprecipitates, radioimmunoassay, oligonucleotide microarrays, antibody microarrays. LSAMP nucleotide and encoded amino acid sequences for exons 1a and 1b are shown in SEQ ID NOs: 1-5. Exon 1a in humans was previously not annotated, but its ortholog in mouse is known. Amino acid and nucleotide sequences which are at least 90%, 95%, 97%, 98%, or 99% identical to the listed sequences may also be used.

As described in detail in the examples, the level of expression of exon 1a (or of a LSAMP transcript which contains exon 1a) is inversely correlated with severity of disease. Thus more severely affected individuals express less LSAMP transcript containing exon 1a. The range of difference between normal individuals and severely affected individuals is greater than 6-fold. The expression of exon 1b appears to be relatively constant. Expression levels can be determined in any tissue which expresses LSAMP, preferably in a cardiovascular tissue. Other tissues which express LSAMP and in which expression can be tested include lung, kidney, prostate, small intestine, spleen, thymus, uterus, fetal brain, and placenta.

Test cells for screening compounds can be human or other mammalian cells, including but not limited to mouse cells. The cells can be from any tissue type, including but not limited to smooth muscle cells, aorta cells, lung, kidney, prostate, small intestine, spleen, thymus, uterus, fetal brain, or placenta. The cells can be, for example, in culture or can be tissue explants.

Test compounds can be purified single compounds, racemic mixtures, mixtures of compounds, single enantiomers, natural products, synthetic products, members or groups of members of combinatorial libraries. The test compounds can have a known pharmacological activity or they can have no previously known pharmacological activity. Since the screening method relies on activity, there is no necessity for pre-screening or selecting compounds that have particular structures or properties. However, pre-screening or selecting is not precluded.

In vitro transcription and coupled in vitro transcription/translation systems are known in the art and can be selected by the skilled artisan as desired. Components which will be typically used are ribonucleotide triphosphates, RNA polymerase, and appropriate buffers and co-factors. The ribonucleotides triphosphates may optionally be labeled to render them readily detectable and quantifiable. Cell-free translation systems, such as extracts of rabbit reticulocytes, wheat germ and Escherichia coli can be optionally used. These extracts typically contain ribosomes, tRNAs, aminoacyl-tRNA synthetases, initiation, elongation and termination factors, etc. These may be supplemented with amino acids, energy sources (ATP, GTP), energy regenerating systems (creatine phosphate and creatine phosphokinase for eukaryotic systems, and phosphoenol pyruvate and pyruvate), and other co-factors (Mg⁺², K⁺, etc.). If translation is carried out, then the amino acids can be labeled and quantified. Translation product can be measured as a means of measuring transcription product.

Any gene can be used as a comparator to exon 1a of LSAMP so long as it is expressed at a relatively constant amount throughout the cell cycle and it is consistently expressed in the particular cells or tissues being tested. Such genes are typically thought of as “housekeeping” genes. Exemplary of such genes is GAPD, which encodes glyceraldehyde phosphate dehydrogenase. Other “housekeeping” genes can be used as is convenient for the practitioner. Other such genes include, but are not limited to RRN18S (18S ribosomal RNA); ACTB (Actin, beta); PGK1 (Phosphoglycerate kinase 1); PPIA (Peptidylprolyl isomerase A; cyclophilin A); RPL13A (Ribosomal protein L13a); RPLPO (Ribosomal protein, large, P0); B2M (Beta-2-microglobulin); YWHAZ (Tyrosine 3-monooxygenase/tryptophan 5-monooxygenase activation protein, zeta polypeptide); SDHA (Succinate dehydrogenase); TFRC (Transferrin receptor; p90, CD71); ALAS1 (Aminolevulinate, delta-, synthase 1); GUSB (Glucuronidase, beta); HMBS (Hydroxymethyl-bilane synthase); HPRT1 (Hypoxanthine phosphoribosyltransferase 1); TBP (TATA box binding protein); and TUBB (Tubulin, beta polypeptide).

Polymorphisms which have been identified as linked to the LSAMP gene, in particular as linked to the intron between exons 1a and 1b, can be used to test people for their risk of cardiovascular genes. Suitable polymorphisms include but are not limited to the G allele of SNP rs 1875518 and the A allele of SNP rs1767232. A polymorphism can be identified in a family member (proband) and then traced through other members of the family. Identifying a linked polymorphism in an individual or in a family member will increase the level of scrutiny and monitoring in otherwise risk-free or low-risk individuals. Preventive treatments may also be applied.

Previously, LSAMP was known as a neuronal surface glycoprotein found in cortical and subcortical regions of the limbic system. During development of the limbic system, this encoded protein was found on the surface of axonal membranes and growth cones, where it was thought to act as a selective homophilic adhesion molecule and to guide the development of specific patterns of neuronal connections. It was not implicated in either normal or pathologic heart function.

A determination of risk based on one of the methods of the present invention, e.g., genetic marker or expression testing, need not be used in isolation from other traditional cardiovascular risk factors. The risk determined by the present invention appears to be independent of other risk factors. Thus, one or more risk factors can be assessed and weighed in determining a course of treatment or monitoring. Other factors which can be considered include without limitation triglycerides, cholesterol, high blood cholesterol, diabetes mellitus, hypertension, family history, and cigarette smoking. Other evaluations which can optionally be performed in conjunction with one or more of the present invention include family history evaluations, echocardiograms, stress tests, blood pressure measurements, ejection fraction measures, etc.

Antibodies according to the present invention can be monoclonal or polyclonal. Methods of generating antibodies which specifically bind to a particular protein are well known in the art. The first step in any such method is inoculation of an animal, such as a mouse, goat, or rabbit, with a preparation that comprises the antigen of interest. Adjuvants can be administered, as is known in the art. Polyclonal antibodies can be obtained from the blood of an inoculated animal. To make monoclonal antibodies, spleen cells are harvested from the inoculated animal and typically fused with myeloma cells to form hybridomas. The hybridomas secrete antibodies, which can be collected and tested for the desired specificity. According to the present invention, an isolated antibody composition specifically binds to a human LSAMP protein comprising an exon 1a encoded sequence, such as that shown in SEQ ID NO: 2 (exon 1a). Preferably the antibody composition does not specifically bind to a human LSAMP protein comprising an exon 1b encoded sequence, such as that shown in SEQ ID NO: 5 (exon 1b). Thus the antibodies can be used to distinguish between these two forms of LSAMP protein. Desirably the difference in binding between the two forms of LSAMP protein will be at least 10-fold, at least 20-fold, at least 50-fold, or at least 100-fold. If a polyclonal antibody composition is used, it can be depleted of antibodies which bind to LSAMP protein comprising an exon 1b encoded sequence using, for example, a column comprising LSAMP protein comprising an exon 1b-encoded sequence. Other methods for depletion of antibodies with undesirable binding properties are known in the art and can be used as is convenient. Monoclonal antibodies can be screened and selected for one which has the desired binding properties, as discussed above.

A number of different kits are provided by the present invention for carrying out the prognostic methods disclosed herein. The kits may provide all or a subset of the reagents that are required for practicing the invention. The kits may comprise written instructions, in paper or electronic form, or a reference to an on-line set of instructions. The instructions may contain data from a population of affected and/or control individuals, against which the results determined using the kit can be compared. Containers which hold the components of any given kit can vary. The kits may be divided into compartments or contain separate vessels for each component. The components may be mixed together or may be separated. Optional components of the kit may include means for collecting, processing, and/or storing test samples. Control samples may also be optionally included in the kits. One kit of the present invention includes an antibody. The antibody specifically binds to an LSAMP protein comprising a sequence as shown in SEQ ID NO: 2 (exon 1a) but does not bind to a protein comprising a sequence as shown in SEQ ID NO: 5 (exon 1b). Any such antibody as discussed above may be used. The antibody may comprise a label or may be linked to a solid support. Such labels or supports facilitate detection. The kit may optionally comprise an antibody which specifically binds to a housekeeping gene product, such as GAPD. Such an antibody can be used to normalize results obtained with the antibodies which bind to the analyte.

Another type of kit contains a pair of primers for amplifying a single nucleotide polymorphism (SNP) marker. The SNP marker is linked to the LSAMP gene. Linked markers are within 50, 100, 150, 200, or 300 kb of the LSAMP gene. The SNP marker can be, for example, rs1676232 or rs1875518. The primers for amplifying hybridize to and preferably are complementary to the sequences which flank the SNP. In order to hybridize sufficiently for amplification, the primers are at least 95%, 97%, 98%, or 99%, identical to the flanking sequences. Flanking sequences of markers rs1676232 and rs1875518 are provided in SEQ ID NO: 11-14. The kit may also contain a probe that hybridizes to the SNP marker and which includes an A or G at the polymorphic single nucleotide or which has its 3′ terminus at the nucleotide immediately adjacent to the polymorphic single nucleotide. Like the primers, the probes are at least 95%, 97%, 98%, or 99%, identical to the SNP marker sequence in order to hybridize specifically and efficiently. Primers and probes are at least 12, 14, 16, 18, 20, 22, or 25 nucleotides in length to ensure sufficient homology for hybridization and specificity. Another optional component of the kit is a mixture of or individual ddNTPs and dNTPs. These can be used, e.g., for a single nucleotide primer extension reaction to determine which nucleotide is present at the SNP. DNA polymerases for amplification of genomic sequences and other enzymes may also be included in the kit.

Still another type of kit contains a forward and a reverse primer for amplifying a human LSAMP cDNA which comprises exon 1a as components. The forward and reverse primers hybridize to opposite strands of a cDNA and have 3′ ends which converge when extended. Primers typically comprise at least 12, 14, 16, 18, 20, or 22 contiguous nucleotides selected from contiguous nucleotides of SEQ ID NO: 1 and 3. This kit can be used to quantify expression of LSAMP transcript that comprises exon 1a. Reverse transcriptase, DNA polymerase, and dNTPs may be included in the kit. Control primers for amplifying a housekeeping gene's transcript may also be included in the kit.

A cDNA which encodes all or part of an LSAMP protein according to SEQ ID NO: 8 may, e.g., comprise all of an LSAMP protein coding sequence or only that portion encoded by exon 1. Portions that are at least 18 contiguous nucleotides of exon 1a of LSAMP according to SEQ ID NO: 1 or 3 can be used as probes or primers to measure exon 1a expression. Such portions can also be used to express an immunogen for generating antibodies to LSAMP protein encoded by a transcript which includes exon 1a. The cDNA can be isolated or it can be in a DNA vector, for replication and or expression purposes. The vector may be in a host cell, mammalian, bacterial, insect, yeast, or other useful families, genuses, or species. Suitable vectors and host cells are known in the art for a variety of purposes and can be selected as needed or desired for a particular purpose.

An isolated and purified LSAMP protein which comprises a portion encoded by nt 298-365 of exon 1a (SEQ ID NO: 1) is also provided. Isolated and purified proteins are typically removed from cells. The level of purity may be at least 1%, 5%, 10%, 25%, 33%, 50%, 75%, or 90%. Purification may be achieved by any method known in the art, including but not limited to immunopurification methods, such as immunoaffinity columns. The LSAMP protein will have a sequence which is at least at least 95%, 97%, 98%, or 99% identical to the amino acid sequence shown in SEQ ID NO: 8. The variation in sequence will accommodate different allelic forms of the protein which are found in the human population.

One or more aspects of the invention may be embodied in computer-executable instructions, such as in one or more program modules, executed by one or more computers or other devices. Generally, program modules include routines, programs, objects, components, data structures, etc. that perform particular tasks or implement particular abstract data types when executed by a processor in a computer or other device. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like.

About 20% of all cardiovascular events occur in individuals that have no identified traditional cardiovascular risk factors (20). The lack of effect on the association by adjusting for known CAD risk factors suggests that the risk conferred by the novel locus reported here is in addition to traditional CAD risk factors. This observation supports our previous findings on the GENECARD dataset, showing that the families contributing to the linkage evidence on the chromosome 3q13 locus have lower triglycerides/cholesterol levels and fewer other known risk factors (Shah et al, submitted). Furthermore, the risk associated with LMD is so pronounced that it dominates competing risks, such as those associated with CABG (21). Thus the identification of asymptomatic individuals at high risk for LMD could have a significant impact on the application of preventive and therapeutic intervention, as by conventional standards of therapy this cohort of patients may normally go untreated, often presenting for medical attention only after their first cardiac event, or post-mortem due to sudden cardiac death. The polymorphism reported here, rs1676232, is a powerful risk marker, and estimated to explain 34% (95% CI: 12 to 55%) of LMD in this sample of patients.

Ethnic differences in CAD risk factors are well known. Thus, while the African-American sample size remains small, it is worth noting that the association became stronger when the African-American dataset was added to the larger Caucasian dataset, suggesting that this novel locus is affecting both ethnic groups in a similar manner. This finding also suggests that this locus represents a major gene influencing CAD risk.

Our study demonstrates the power of “genomic approaches.” LSAMP has never been implicated in cardiovascular biology prior to this study, and thus would have been missed through a candidate gene approach. LSAMP is a 64-68 kilodalton cell membrane glycoprotein (22) and has been shown to mediate cell-cell adhesion in neurons (23; 24). It is believed to represent a selective guidance cue in the development of limbic and thalamocortical neuronal systems (25). Over-expression of LSAMP in renal cell carcinoma (RCC) lines inhibited cell proliferation (26). It is conceivable that LSAMP mediates cell-cell adhesion and regulates smooth muscle cell proliferation in the vascular wall. Nelovkov et al. has suggested LSAMP is involved in behavioral responses to adverse environments (25), and it could be regulating similar responses to environmental stimuli in the arterial wall as well. Further study is warranted to understand the role of the LSAMP gene in vascular development and remodeling, and why genetic variations in LSAMP manifest particularly in LMD.

Comparative genomics have shown several highly conserved sequence blocks between human and mouse/rat/chicken in the large alternative intron 1 of LSAMP gene (see the website at the domain ensembl.org). Conserved intergenic sequences are believe to be more likely to contain cis-regulatory elements or motifs with functional features (27; 28). The SNP rs1676232, whose genotype correlates with LSAMP_{—}1a mRNA level, resides in one of these highly conserved blocks. Although long-range gene regulation is not as intuitive as proximal promoter control, it is not unusual for a cis-regulatory element to operate over long distance (29-31). For example, in the genetic study of preaxial polydactyl), it has been found that disruption of a cis-element located 1 Mb upstream of the shh gene leads to ectopic expression of the gene (32). In a recent study, Nobrega and colleagues demonstrated cis-regulatory sites exist in regions kilobases away from the transcription start site of the target gene (33). The prospective mechanisms for the long-range control include distance-independent enhancers, chromatin remodeling through epigenetic alterations such as methylation. In fact, both alternative promoters of LSAMP contain CpG islands. It has already been shown that LSAMP_{—}1b expression is methylation sensitive in RCC tumors (26). We have recently reviewed the potential role of epigenetics in arteriosclerosis (34).

The absence of a primary age-of-onset effect was unexpected. It suggests that additional loci (either primary or modifier genes) exist that contribute to early-onset disease CAD. Indeed, modifier genes affecting age-of-onset have been discovered for both Parkinson and Alzheimer disease (35; 36) and seem likely to be involved in the complex phenotype of cardiovascular disease as well.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Methods

### Subjects

CATHGEN subjects were recruited through the cardiac catheterization laboratories at Duke University Hospital (Durham, N.C.) with approval from the Duke Institutional Review Board. All subjects undergoing catheterization were offered participation in the study and signed informed consent. Medical history and clinical data were collected and stored in the Duke Information System for Cardiovascular Care database maintained at the Duke Clinical Research Institute (10). GENECARD subjects have been described previously (11).

### Classification Criteria

Two case groups were identified for initial screening: 1) young affected (YA_aac) with a CAD index (CADi)>32 and the age-at-catheterization (AAC)<56 years, and 2) old affected (OA_aac) with a CADi >67 (a higher threshold was used in older patients to adjust for the higher baseline extent of CAD in this group) and AAC ≧56 years. The CADi is a numerical summary of coronary angiographic data that incorporates the extent and anatomic distribution of coronary disease (Table 1) (12). CADi has been shown to be a better predictor of clinical outcome than the extent of CAD (13). Controls had an AAC >60 years with a CADi≦23 and no documented cerebrovascular or peripheral vascular disease, myocardial infarction (MI), or interventional cardiac procedures. To further ensure the accuracy of the age data in the CATHGEN dataset, medical records were reviewed to determine the age-of-onset (AOO) of CAD, i.e. the age at first documented surgical or percutaneous coronary revascularization procedure, MI, or cardiac catheterization meeting the above defined CADi thresholds. The CATHGEN case groups were also reclassified into young affected (YA_aoo, AOO <56 years) and old affected (OA_aoo, AOO ≧56 years) based on AOO.

TABLE 1
Definition of the coronary artery disease index (CADi) (12)

| Extent of CAD | CADi |
|---|---|
| No CAD ≧50% | 0 |
| One-VD 50% to 74% | 19 |
| One-VD 75% | 23 |
| One-VD ≧95% | 32 |
| Two-VD | 37 |
| Two-VD (both ≧95%) | 42 |
| One-VD ≧95%, proximal (LAD) | 48 |
| Two-VD ≧95% LAD | 48 |
| Two-VD ≧95% proximal LAD | 56 |
| Three-VD | 56 |
| Three-VD ≧95% in at least one vessel | 63 |
| Three-VD 75% proximal LAD | 67 |
| Three-VD ≧95% proximal LAD | 74 |
| Left main (75%) | 82 |
| Left main (≧95%) | 100 |

CAD = coronary artery disease; LAD = left anterior descending coronary artery; VD = vessel disease

Two additional case groups were constructed on the basis of severity of CAD: “severe affected” (SA) and “left main affected” (LM), defined in the CATHGEN dataset as individuals having a CADi >67 and CADi ≧82, respectively, regardless of age. Finally, an independent case dataset was created by including one proband (N=420 individuals) from each of the GENECARD families used in the GENECARD genome screen (9). In the GENECARD dataset, the CADi was not available for all individuals. Therefore, medical records were reviewed to evaluate the CAD severity in GENECARD probands for comparison with CATHGEN cases.

### DNA Pooling, Allelotyping and Genotyping

A DNA pooling strategy was used to initially screen SNPs for association. Pools of approximately 100 individuals were constructed and allelotyping was performed using the method of Hoogendoorn et al (14) with modifications (Supplementary Methods). Individual genotyping was performed using the TaqMan® Allelic Discrimination Assay. If available, Assay-On-Demand assays were used, otherwise primers and probes were designed using the Primer Express software. Vigorous quality controls were implemented to ensure the accuracy of genotyping (Supplementary Methods).

### Gene Expression Analysis:

Human total RNA Master Panel II was purchased from BD biosciences (Palo Alto, Calif.). Normal human aortic endothelial cells and smooth muscle cells were purchased from Cambrex Bio Science, Inc (Walkersville, Md.). Aorta collection and RNA extraction have been previously described (15). Total RNA was used for cDNA synthesis using Advantage™ RT-for-PCR Kit (BD biosciences). Real-time RT-PCR reaction was performed using Taqman® universal PCR master mix, following the manufacture's instructions (AB, Foster City, Calif.). Data were normalized to glyceraldehyde-3-phosphate dehydrogenase (GAPD) expression levels within the same sample.

### Statistical Analysis

Disease association was initially examined using logistic regression analysis adjusted for gender and ethnicity. To adjust for known CAD risk factors, a multivariable logistic regression model was used which included hypertension, diabetes mellitus, body mass index (BMI), dyslipidemia, and smoking history as covariates. Association tests were performed using an additive allele model. Haplotype tagging SNPs were chosen using LdSelect 1.0 (16). The threshold parameters for the correlation coefficient r² and the minor allele frequency (MAF) were set as r²≧0.8 and MAF≧0.1. The Graphical Overview of Linkage Disequilibrium (GOLD) program (17) was used to assess linkage disequilibrium (LD) between SNPs. Haplotype analysis was performed using Haplo Stats 1.1.0 (Mayo Clinic, Rochester, Minn.). Regression analysis was performed to evaluate the relationships between atherosclerosis burden, genotype and gene expression. A mixed model was fit including a random effect for each aorta along with fixed effects for atherosclerosis burden and genotype. An F-test was used to test for differences in gene expression for the fixed effects. SAS 9.0 (SAS, Cary, N.C.) was used for statistical analyses.

### Allelotyping in DNA Pools

DNA samples from 301 YA_aac, 168 OA_aac, and 204 controls were used for the initial pooling studies. Pools of approximately 100 individuals were constructed by mixing 200 ng of DNA from each individual. The YA_aac group had three DNA pools of 100, 100, and 101 individuals, while the OA_aac group had two pools of 84 individuals and the control group had two DNA pools of 102 individuals. Each DNA sample was diluted to approximately 20 ng/ul and the concentration was measured using PicoGreen® dye (Molecular Probe, Inc., Eugene, Oreg.). The final concentration of the DNA pool was adjusted to 10 ng/ul by adding an appropriate volume of deionized water.

Allelotyping was performed using the method of Hoogendoom et al (14) with modifications. Briefly, genomic sequence around a SNP is amplified by the polymerase chain reaction (PCR). A short probe is annealed adjacent to the site of polymorphism and is extended differentially in the presence of appropriate ddNTP and dNTP mixture (primer extension or PE). Finally, the allele-specific extended primers from PE are separated and detected by denaturing high-performance liquid chromatography (DHPLC). The allele frequency (f) is calculated using the peak height (h) of the two extended primers: f=h₁/(h₁+h₂). The procedure was modified in this study by eliminating the unequal amplification factor k (14) used in calculating the corrected allele frequency (f_{corr}): f_{corr}=h₁/(h₁+kh₂), as k is applied in calculating the allele frequency in both case and control pools, and calculation with and without the factor k did not affect the estimation of allele frequency differences between pools (Table 2). The unequal amplification factor k is calculated as k=h₁′/h₂′, where h₁′ and h₂ are peak heights representing two alleles in a heterozygous individual. Identifying the heterozygous individuals and estimating the unequal amplification factor k for each one of SNPs that will be screened translates into extra cost and time. Therefore, elimination of k significantly reduce work load in the modified screening procedure.

TABLE 2
The correction factor k has minor effect on estimation
of allele frequencies between DNA pools.

| Allele frequency* | | |
|---|---|---|
| | Uncorrected | Corrected |
| Expected | PELC | PELC |

rs153477

| | | | |
|---|---|---|---|
| Pool A | 0.340 | 0.456 ± 0.025 | 0.382 ± 0.014 |
| Pool B | 0.290 | 0.415 ± 0.024 | 0.342 ± 0.009 |
| Δf (Pool A − Pool B) | 0.050 | 0.042 ± 0.009 | 0.040 ± 0.009 |

rs483349

| | | | |
|---|---|---|---|
| Pool A | 0.430 | 0.467 ± 0.01 | 0.439 ± 0.006 |
| Pool B | 0.370 | 0.411 ± 0.008 | 0.383 ± 0.002 |
| Δf (Pool A − Pool B) | 0.060 | 0.056 ± 0.004 | 0.055 ± 0.003 |

*Expected allele frequency is calculated by counting genotype assigned by Taqman ® Allelic Discrimination Assay to each individual in the pool. Allele frequencies estimated by primer extension followed by dHPLC (PELC) are reported as mean ± SEM from 4 replicates: uncorrected PELC allele frequency = h₁/(h₁ + h₂); corrected PELC allele frequency = h₁/(h₁ + kh₂)

### PCR and Primer Extension Reaction

Sequences flanking the identified SNPs were retrieved from the NCBI dbSNP database (http://www.ncbi.nlm.nih.gov/SNP/). PCR primers were designed using the Primer 3 program (http://www-genome.wi.mit.edu/cgi-bin/primer/primer3_www.cgi). Primers used for primer extension were manually designed from either upstream or downstream sequences adjacent to the polymorphism site. All primers were synthesized by Integrated DNA Technologies, INC (Coralvill, Iowa) at 25 nmol scale with standard desalt purification. Primer and probe sequences are listed in Table 3. PCR was set up with the following conditions: 18 ng of pooled genomic DNA, 100 μM dNTPs (Invitrogen, Carlsbad, Calif.), 24 μmol of each forward and reverse PCR primers and 0.9 unit of Platinum® Taq DNA Polymerase (Invitrogen) in 30 μl 1×PCR buffer. PCR was performed with an initial denaturation (95° C. for 10 min), followed by 35 cycles (94° C. for 10 sec, 55° C. for 30 sec, and 72° C. for 1 min) and a final extension (72° C. for 10 min). To remove excess PCR primers and dNTPs, 20 μl of PCR reaction was treated with 1 μl of EXOSAP IT (Amersham Bioscience, Piscataway, N.J.) at 37° C. for 60 min, followed by incubation at 80° C. for 15 min to inactivate the enzyme. The primer extension reaction was set up in 25 μl volume with 6.5 μl of purified PCR product, 50 μM of the appropriate ddNTP/dNTP mix, 0.6 pmol/μl of extension primer, 2.5 μl of concentrated Thermo Sequenase buffer, and 0.024 unit/μl Thermo Sequenase (Amersham Bioscience). The primer extension reaction was performed with initial denaturation at 96° C. for 1 min, 75 cycles of 96° C. for 10 sec, 55° C. for 30 sec and 60° C. for 30 sec and final extension at 60° C. for 5 min. All the reactions were carried out in PTC-200 DNA Engine (MJ Research, Watertown, Mass.).

TABLE 3
Primer and probe sequence information for allelotyping in DNA pools.

| SNP | | Primer Sequence | Probe Sequence | ddNTP/dNTP mix |
|---|---|---|---|---|
| rs1401951 | forward | TTGACTGACGTTCTTCCATGA | GACTTGTGCAAGTTAAAACTTGAAA | ddTTP/dA, C, GTP |
| | reverse | AGGGAAAGGGCATATGGAGT | | |
| rs1456186 | forward | TAAGGTTTTCGAGGGGAGGT | GAGTAGCCTGGGGATGAGCAAA | ddGTP/dA, C, TTP |
| | reverse | ATCAGCGAACCTGCTCAAAG | | |
| rs1486336 | forward | TGTTTCTCAGCCAGGGTTGT | CTTTGAATCCCATGATGATAGATTGA | ddGTP/dA, C, TTP |
| | reverse | TGGTTGATCAATGCAAATCC | | |
| rs1499989 | forward | TTGAGAGTGAAGGGGTTTGAG | AGGAAAGCCGTCTGAGGAGGAG | ddGTP/dA, C, TTP |
| | reverse | TTTCCCTTCCAAAACATTGC | | |
| rs1501882 | forward | TGTTCACAGTGGGAGTGTTGG | CTTTAGAATTGTAATGGTCATCTCGAC | ddATP/dC, G, TTP |
| | reverse | GGCATGAAACATATTTGAGGCTTA | | |
| rs1875516 | forward | AGCCAGGCTAACTGTGTTCAAG | CACTTGAGTAAATGGGCAGAAGAT | ddTTP/dA, C, GTP |
| | reverse | GGCCTAAGATGGGGAATGAAAT | | |
| rs1875518 | forward | TTGCCTTACTTTACCTCTTCTGC | CACTTAAGGGTGCTCATTAGGTATTAC | ddCTP/dA, G, TTP |
| | reverse | TTCTGCCCTTAATTTAATGTTGA | | |
| rs1968010 | forward | TGCGGTAATCACTATCCCAAG | AGGAAACAGTGCATTGGGGC | ddCTP/dA, G, TTP |
| | reverse | CATCTTGAAGTGACCCTGGAG | | |
| rs2282171 | forward | CCGAAAGAGGAAATGCTTTG | GGCGGGACCGCGAGTTAA | ddGTP/dA, C, TTP |
| | reverse | ACGACAACCCCTACCATITG | | |
| rs39688 | forward | GGCTTGGTCATGGAAATTGT | GCAGCTTCATCAGATCAAGGACATT | ddTTP/dA, C, GTP |
| | reverse | ATCCTCCCAACCCCTTACTC | | |
| rs483349 | forward | CCGCTGGCTTGTGAATAACT | GTGGCTCCCTACAGTTGGGGTTC | ddCTP/dA, G, TTP |
| | reverse | CCTGAAACTGGGGGTAGTCA | | |
| rs553070 | forward | CCCCATGTCATTTCTACTCCA | GGAAACTTTTGGAATCTCCTATTCATC | ddCTP/dA, G, TTP |
| | reverse | GTGGCATCTTTGGGATCAAT | | |
| rs705233 | forward | CCCAGAATTTTTAGAGAAATCGAA | TATCTTTTCAGCTAATGCATCTTCCA | ddCTP/dA, G, TTP |
| | reverse | TCCTCTGCTGTTATCTTITCAGC | | |
| rs725154 | forward | TGGGAAAGCTTTTTGGATTG | GAAGATAGGAACAGTCACATAGC | ddCTP/ddTTP |
| | reverse | CGTGGTTCTCAGGTAGGACA | | |
| rs812824 | forward | ACAGTACACAGGCACCCACA | GTGTTCCAGGGCATTAATTGTGTC | ddCTP/dA, G, TTP |
| | reverse | TTTTTCTGTGATTTGAGATTGITCTT | | |
| rs843855 | forward | TTTTATGGCCAAAGCCAGTC | CCATGACAGGAATGTGGATATACA | ddTTP/dA, C, GTP |
| | reverse | GGGGTGTTTGGGTAAGAATG | | |

Primers are SEQ ID NO: 17-48, respectively. Probes are SEQ ID NO: 49-64, respectively.

### Denaturing HPLC Analysis

Allele-specific extended primers from the primer extension reaction were analyzed by DHPLC on a WAVE DNA Fragment Analysis System (Transgenomic, Omaha, Nebr.) using DNAsep®HT cartridge. The eluent buffer was composed of 82%-20% of buffer A (0.1 M triethylamine acetate buffer (TEAA), pH 7.4) and 18% to 80% of buffer B (25% acetonitrile in 0.1 M TEAA, pH 7.4) at a constant pump flow rate of 1.5 ml/min. During the analytical run, the oven temperature was set at 70° C. to keep the oligonucleotides denatured. Once eluted, the extended primers were measured by a UV detector at 260 nm. For each SNP examined in this study, all reactions and DHPLC analysis on the different pools were conducted at same time. Each pool was alleotyped three times and the mean was used for the final estimates of the allele frequency difference between pools.

Statistical Analysis for Allelotyping Data

For the DNA pooling data, we used the z-test for 2 independent proportions.

$$z = \frac{( {p}_{1} - {p}_{2} )}{\sqrt{( \frac{1}{2 {n}_{1}} + \frac{1}{2 {n}_{2}} ) p ( 1 - p ) + {σ}_{exp}^{2}}} where p = \frac{( {n}_{1} {p}_{1} + {n}_{2} {p}_{2} )}{{n}_{1} + {n}_{2}}$$

Where pⱼ represents the mean allele frequency in group j (j=1 or 2) and nⱼ is the total number of subjects in each group. σ²ₑₓₚ is the variance due to the pooling experiment estimated as described below. The p-value for the z-test was estimated using the standard normal probability tables. The sources of variation in the estimation of pool allele frequency were evaluated using analysis of variance for each SNP. The mean standard error (MSE) for variability among the repeated measurements of each SNP was estimated by including a fixed effect for case and control groups and a fixed effect for the pool nested within group. We used an adjusted MSE as the estimate of the experimental variability, i.e. σ²ₑₓₚ, in estimating the DNA pool allele frequency. The experimental variability among the repeated measurements of allele frequency differences between DNA pools ranged from 0.001 to 0.0001 with a mean at 0.0005 (data not shown).

### SNP Genotyping

SNPs were genotyped using the Taqman® Allelic Discrimination Assay in a 384-well format following manufacturer's instruction. For the purpose of quality control, one blank, two Centre d'Etude Polymorphisme Humain (CEPH) pedigree individuals (38) and nine quality control samples were included for every quadrant of the 384-well plate. In total, 32 quality control samples were used to provide duplicated samples within one quadrant, across quadrants within one plate, and across plates. Results of the CEPH and quality control samples were compared to identify possible sample plating errors and genotype calling inconsistencies. Hardy-Weinberg equilibrium (HWE) testing was performed for all markers. SNPs that showed mismatches on quality control samples or that failed the HWE test (p<0.05) in controls were reviewed by an independent genotyping supervisor for potential genotyping errors. All SNPs examined were successfully genotyped for 95% or more of the individuals in the study. Error rate estimates for SNPs meeting the quality control benchmarks (based on over 26,000 duplicate genotypes) were less than 0.2%.

### Example 2

### Identification of significant linkage

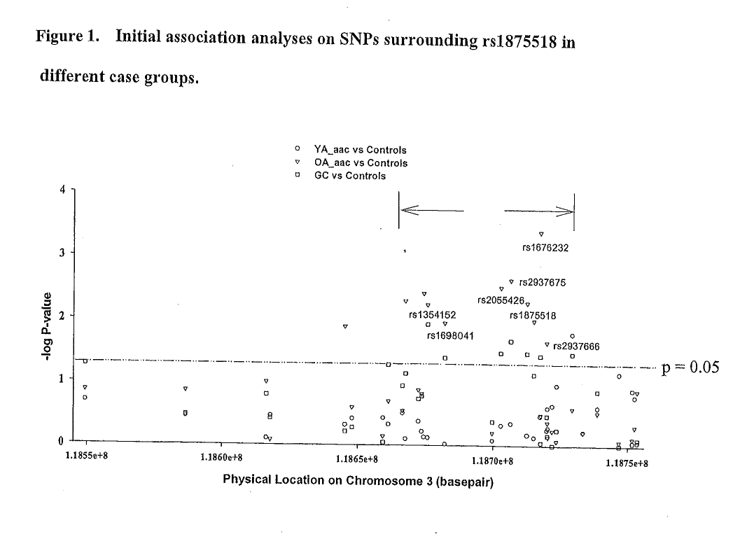

From 2000 subjects enrolled in CATHGEN, 469 cases and 204 controls were selected for this study (Table 4). Initially, we allelotyped 16 SNPs at 150 kilobase (Kb) intervals across a three megabase (Mb) region surrounding D3S2460, the linkage peak marker (FIG. 1). This test screening found a significant allele frequency difference between OA_aac and controls (Δ=12.2%, p=0.001) for rs1875518 (A/G) (Supplementary FIG. 1). This was confirmed by genotyping, showing that the frequency of the G allele is 12.6% higher in the OA_aac group than controls. None of the other 15 SNPs showed evidence of association.

TABLE 4
Clinical characteristics of CATHGEN cases and controls.

| | | | | | Severe | | |
|---|---|---|---|---|---|---|---|
| | YA_aac | OA_aac | YA_aoo | OA_aoo | Affected | Left Main | Control |
| Number of | 301 | 168 | 358 | 111 | 202 | 120 | 204 |
| individuals | | | | | | | |
| Age-at-cathe- | 49.2 (5.7)* | 69.4 (8.2) | 51.5 (7.7)* | 72.5 (7.6) | 66.1 (10.7)* | 65.4 (11.0)* | 70.5 (6.9) |
| terization, | | | | | | | |
| mean (SD) | | | | | | | |
| Age-of-onset, | 45.5 (6.7) | 60.3 (10.5) | 46.1 (6.5) | 66.1 (7.6) | 57.4 (12.0) | 56.5 (12.3) | N/A |
| mean (SD) | | | | | | | |
| CAD index, | 49.9 (17.6)* | 82.3 (9.6)* | 55.2 (20.7)* | 81.8 (9.1)* | 82.6 (9.8)* | 88.5 (8.7)* | 9.6 (10.8) |
| mean (SD) | | | | | | | |
| Gender, | 78.4%* | 75.0%* | 79.9%* | 68.5%* | 76.2%* | 72.5%* | 44.6% |
| % Male | | | | | | | |
| Caucasian, % | 68.8% | 85.7%* | 70.4% | 89.2%* | 83.2% | 85.0% | 73.0% |
| BMI, | 31.1 (6.6)* | 28.7 (6.3) | 30.7 (6.5)* | 28.7 (6.8) | 29.2 (6.3) | 29.0 (6.0) | 28.3 (6.7) |
| mean (SD) | | | | | | | |
| Ever- | 70.4%* | 59.5%* | 70.1%* | 55.0% | 60.4%* | 60.0%* | 40.7% |
| smoked, % | | | | | | | |
| Diabetes, % | 33.6%* | 32.1%* | 33.0%* | 33.3%* | 33.2%* | 32.5%* | 15.2% |
| Hyperten- | 63.5% | 80.4%* | 65.6% | 82.0%* | 81.2%* | 80.8%* | 68.6% |
| sion, % | | | | | | | |
| Dyslip- | 68.4%* | 75.0%* | 71.8%* | 67.6%* | 76.2%* | 77.5%* | 42.7% |
| idemia, % | | | | | | | |
| History | 50.8% | 48.8% | 53.6% | 38.7% | 48.5% | 45.0% | N/A |
| of MI, % | | | | | | | |

*Significant difference between cases and controls (p < 0.05). Analysis of variance was performed by Chi-square tests for categorical variables and t-tests for numeric variables.
N/A, not available.

### Example 3

### Genotyping Surrounding Linkage Marker

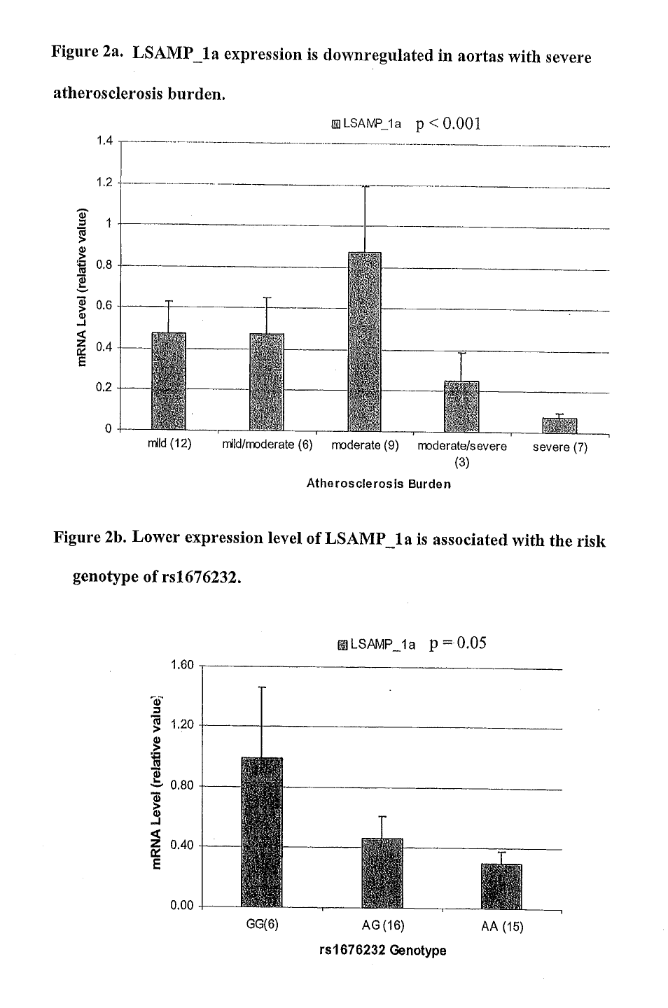

Due to this significant finding, we ceased our pooling screen and began genotyping SNPs surrounding rs1875518 at a high density. Since there is no annotated gene within one Mb of rs1875518 (http://www.ensembl.org, Human v27.35a.1), 35 SNPs were chosen over a 200 kilobase (kb) “non-genic” region (FIG. 1c). Using the logistic regression model adjusting for gender and ethnicity, several SNPs showed evidence of association (p<0.05) in the OA_aac group with the strongest association at rs1676232 (p<0.001), while only rs2937666 was associated (p=0.017) with the YA_aac group (FIG. 2 and Table 5a). The association observed in the OA_aac, but not in the YA_aac group, persisted even after adjustment for traditional CAD risk factors (Table 5b). A similar pattern of association was observed when the analyses were performed in Caucasians only (data not shown), suggesting that both Caucasian and African-American groups had similar association characteristics at this locus. Most importantly, GENECARD probands also gave a positive association at rs1354152, rs1698041, rs2055426, rs2937675, rs1875518, rs1676232, and rs2937666 for CAD (p<0.05), confirming the observations in the CATHGEN dataset (FIG. 2 and Table 6a and Table 6b).

TABLE 5a
Association tests between case groups and controls in
the basic model adjusting for gender and ethnicity.

| | YA_aac | OA_aac | YA_aoo | OA_aoo | SA | LM | GC |
|---|---|---|---|---|---|---|---|
| SNP | (N = 301) | (N = 168) | (N = 358) | (N = 111) | (N = 202) | (N = 120) | (N = 420) |
| rs1513172 | 0.324 | 0.293 | 0.143 | 0.902 | 0.262 | 0.211 | 0.883 |
| rs6438389 | 0.541 | 0.288 | 0.641 | 0.120 | 0.284 | 0.370 | 0.254 |
| rs1513156 | 0.205 | 0.139 | 0.227 | 0.120 | 0.085 | 0.052 | 0.053 |
| rs11716267 | 0.358 | 0.139 | 0.845 | 0.479 | 0.310 | 0.141 | 0.335 |
| rs1398626 | 0.786 | 0.101 | 0.517 | 0.147 | 0.253 | 0.611 | 0.159 |
| rs1513162 | 0.349 | 0.827 | 0.557 | 0.986 | 0.770 | 0.699 | 0.378 |
| rs4075039 | 0.475 | 0.013 | 0.261 | 0.051 | 0.024 | 0.003 | 0.608 |
| rs7427839 | 0.379 | 0.252 | 0.462 | 0.074 | 0.134 | 0.068 | 0.527 |
| rs6790819 | 0.375 | 0.729 | 0.511 | 0.780 | 0.680 | 0.752 | 0.903 |
| rs4356827 | 0.465 | 0.201 | 0.529 | 0.041 | 0.139 | 0.086 | 0.052 |
| rs2927275 | 0.308 | 0.283 | 0.488 | 0.046 | 0.175 | 0.205 | 0.113 |
| rs1698042 | 0.802 | 0.005 | 0.528 | 0.019 | 0.010 | 0.023 | 0.072 |
| rs1501881 | 0.418 | 0.132 | 0.554 | 0.131 | 0.172 | 0.028 | 0.182 |
| rs1910040 | 0.609 | 0.152 | 0.734 | 0.144 | 0.195 | 0.061 | 0.165 |
| rs1501885 | 0.739 | 0.004 | 0.503 | 0.004 | 0.005 | <0.001 | N/A |
| rs1354152 | 0.754 | 0.006 | 0.546 | 0.006 | 0.008 | <0.001 | 0.012 |
| rs1698041 | 0.955 | 0.011 | 0.700 | 0.013 | 0.015 | <0.001 | 0.040 |
| rs11713954 | 0.834 | 0.625 | 0.944 | 0.737 | 0.582 | 0.250 | 0.417 |
| rs2055426 | 0.481 | 0.003 | 0.290 | 0.005 | 0.003 | <0.001 | 0.033 |
| rs2937675 | 0.445 | 0.002 | 0.267 | 0.004 | 0.003 | <0.001 | 0.021 |
| rs1875518 | 0.676 | 0.005 | 0.435 | 0.010 | 0.007 | <0.001 | 0.034 |
| rs2937673 | 0.745 | 0.010 | 0.520 | 0.012 | 0.010 | <0.001 | 0.074 |
| rs1676232 | 0.342 | <0.001 | 0.171 | 0.001 | <0.001 | <0.001 | 0.037 |
| rs4855952 | 0.906 | 0.327 | 0.683 | 0.574 | 0.494 | 0.930 | 0.894 |
| rs1501874 | 0.563 | 0.022 | 0.828 | 0.094 | 0.059 | 0.100 | 0.337 |
| rs2937670 | 0.250 | 0.426 | 0.195 | 0.762 | 0.201 | 0.063 | 0.696 |
| rs9824498 | 0.522 | 0.719 | 0.549 | 0.745 | 0.874 | 0.951 | N/A |
| rs1979868 | 0.227 | 0.574 | 0.339 | 0.661 | 0.742 | 0.624 | 0.988 |
| rs1381801 | 0.110 | 0.822 | 0.192 | 0.675 | 0.763 | 0.765 | 0.565 |
| rs2937666 | 0.017 | 0.258 | 0.032 | 0.177 | 0.267 | 0.984 | 0.035 |
| rs1910044 | 0.596 | 0.604 | 0.670 | 0.581 | 0.567 | 0.776 | N/A |
| rs4855955 | 0.244 | 0.297 | 0.346 | 0.219 | 0.317 | 0.109 | 0.135 |
| rs6778437 | 0.071 | 0.856 | 0.098 | 0.877 | 0.610 | 0.979 | 0.989 |
| rs6795971 | 0.130 | 0.766 | 0.174 | 0.776 | 0.976 | 0.641 | 0.884 |
| rs1393192 | 0.167 | 0.493 | 0.258 | 0.368 | 0.915 | 0.576 | N/A |
| rs1466416 | 0.879 | 0.132 | 0.807 | 0.950 | 0.092 | 0.165 | 0.797 |

TABLE 5b
Association tests between case groups and controls in the full model adjusting for gender,
ethnicity, hypertension, body mass index, diabetes, dyslipidemia, and smoking history.

| | YA_aac | OA_aac | YA_aoo | OA_aoo | SA | LM | GC |
|---|---|---|---|---|---|---|---|
| SNP | (N = 301) | (N = 168) | (N = 358) | (N = 111) | (N = 202) | (N = 120) | (N = 420) |
| rs1513172 | 0.693 | 0.453 | 0.400 | 0.972 | 0.365 | 0.381 | 0.336 |
| rs6438389 | 0.131 | 0.148 | 0.137 | 0.076 | 0.158 | 0.344 | 0.176 |
| rs1513156 | 0.019 | 0.041 | 0.017 | 0.053 | 0.036 | 0.058 | 0.013 |
| rs11716267 | 0.348 | 0.300 | 0.835 | 0.798 | 0.506 | 0.264 | 0.519 |
| rs1398626 | 0.854 | 0.024 | 0.476 | 0.045 | 0.051 | 0.216 | 0.106 |
| rs1513162 | 0.436 | 0.702 | 0.683 | 0.899 | 0.938 | 0.735 | 0.334 |
| rs4075039 | 0.972 | 0.074 | 0.680 | 0.152 | 0.126 | 0.029 | 0.510 |
| rs7427839 | 0.443 | 0.732 | 0.539 | 0.353 | 0.444 | 0.347 | 0.720 |
| rs6790819 | 0.315 | 0.588 | 0.450 | 0.805 | 0.861 | 0.767 | 0.506 |
| rs4356827 | 0.413 | 0.176 | 0.362 | 0.091 | 0.145 | 0.171 | 0.116 |
| rs2927275 | 0.209 | 0.144 | 0.260 | 0.048 | 0.091 | 0.208 | 0.112 |
| rs1698042 | 0.383 | 0.014 | 0.233 | 0.016 | 0.017 | 0.023 | 0.430 |
| rs1501881 | 0.467 | 0.255 | 0.595 | 0.278 | 0.336 | 0.106 | 0.222 |
| rs1910040 | 0.816 | 0.142 | 0.952 | 0.219 | 0.190 | 0.136 | 0.081 |
| rs1501885 | 0.755 | 0.028 | 0.542 | 0.028 | 0.036 | 0.005 | N/A |
| rs1354152 | 0.768 | 0.041 | 0.571 | 0.037 | 0.053 | 0.007 | 0.080 |
| rs1698041 | 0.949 | 0.094 | 0.721 | 0.097 | 0.122 | 0.021 | 0.137 |
| rs11713954 | 0.430 | 0.689 | 0.617 | 0.602 | 0.618 | 0.819 | 0.878 |
| rs2055426 | 0.563 | 0.033 | 0.371 | 0.039 | 0.038 | 0.004 | 0.116 |
| rs2937675 | 0.503 | 0.027 | 0.329 | 0.029 | 0.031 | 0.004 | 0.087 |
| rs1875518 | 0.650 | 0.054 | 0.463 | 0.058 | 0.064 | 0.007 | 0.122 |
| rs2937673 | 0.800 | 0.075 | 0.615 | 0.061 | 0.082 | 0.010 | 0.244 |
| rs1676232 | 0.123 | 0.001 | 0.054 | 0.002 | 0.001 | <0.001 | 0.041 |
| rs4855952 | 0.614 | 0.347 | 0.404 | 0.550 | 0.528 | 0.998 | 0.724 |
| rs1501874 | 0.451 | 0.088 | 0.706 | 0.214 | 0.239 | 0.195 | 0.644 |
| rs2937670 | 0.109 | 0.178 | 0.096 | 0.337 | 0.077 | 0.013 | 0.906 |
| rs9824498 | 0.720 | 0.353 | 0.659 | 0.471 | 0.365 | 0.388 | N/A |
| rs1979868 | 0.187 | 0.723 | 0.221 | 0.518 | 0.856 | 0.719 | 0.235 |
| rs1381801 | 0.227 | 0.734 | 0.269 | 0.894 | 0.658 | 0.768 | 0.118 |
| rs2937666 | 0.080 | 0.171 | 0.110 | 0.184 | 0.262 | 0.961 | 0.083 |
| rs1910044 | 0.551 | 0.580 | 0.543 | 0.794 | 0.651 | 0.648 | N/A |
| rs4855955 | 0.550 | 0.154 | 0.640 | 0.073 | 0.148 | 0.037 | 0.552 |
| rs6778437 | 0.284 | 0.940 | 0.276 | 0.894 | 0.722 | 0.688 | 0.575 |
| rs6795971 | 0.429 | 0.712 | 0.431 | 0.588 | 0.925 | 0.411 | 0.601 |
| rs1393192 | 0.282 | 0.226 | 0.379 | 0.105 | 0.446 | 0.248 | N/A |
| rs1466416 | 0.542 | 0.205 | 0.683 | 0.951 | 0.163 | 0.238 | 0.588 |

YA_aac = young affected (CADi >23, age-at-catheterization <56); OA_aac = old affected (CADi >67, age-at-catheterization >=56); YA_aoo = young affected (CADi >23, age-of-onset <56); OA_aoo = old affected (CADi >67, age-of-onset >=56); SA = severe affected (CADi >67, regardless of age-of-onset); LM = left main affected (CADi ≧82, regardless of age-of-onset); GC = GENECARD probands. All the case groups were compared to CATHGEN controls (N = 204) using additive allele model. P-values < 0.05 are in bold.
N/A, data was not available.

TABLE 6a
Association tests between case groups and controls in
the basic model adjusting for gender and ethnicity.

| | YA_aac | OA_aac | YA_aoo | OA_aoo | SA | LM | GC |
|---|---|---|---|---|---|---|---|
| SNP | (N = 301) | (N = 168) | (N = 358) | (N = 111) | (N = 202) | (N = 120) | (N = 420) |
| rs1513172 | 0.324 | 0.293 | 0.143 | 0.902 | 0.262 | 0.211 | 0.883 |
| rs6438389 | 0.541 | 0.288 | 0.641 | 0.120 | 0.284 | 0.370 | 0.254 |
| rs1513156 | 0.205 | 0.139 | 0.227 | 0.120 | 0.085 | 0.052 | 0.053 |
| rs11716267 | 0.358 | 0.139 | 0.845 | 0.479 | 0.310 | 0.141 | 0.335 |
| rs1398626 | 0.786 | 0.101 | 0.517 | 0.147 | 0.253 | 0.611 | 0.159 |
| rs1513162 | 0.349 | 0.827 | 0.557 | 0.986 | 0.770 | 0.699 | 0.378 |
| rs4075039 | 0.475 | 0.013 | 0.261 | 0.051 | 0.024 | 0.003 | 0.608 |
| rs7427839 | 0.379 | 0.252 | 0.462 | 0.074 | 0.134 | 0.068 | 0.527 |
| rs6790819 | 0.375 | 0.729 | 0.511 | 0.780 | 0.680 | 0.752 | 0.903 |
| rs4356827 | 0.465 | 0.201 | 0.529 | 0.041 | 0.139 | 0.086 | 0.052 |
| rs2927275 | 0.308 | 0.283 | 0.488 | 0.046 | 0.175 | 0.205 | 0.113 |
| rs1698042 | 0.802 | 0.005 | 0.528 | 0.019 | 0.010 | 0.023 | 0.072 |
| rs1501881 | 0.418 | 0.132 | 0.554 | 0.131 | 0.172 | 0.028 | 0.182 |
| rs1910040 | 0.609 | 0.152 | 0.734 | 0.144 | 0.195 | 0.061 | 0.165 |
| rs1501885 | 0.739 | 0.004 | 0.503 | 0.004 | 0.005 | <0.001 | N/A |
| rs1354152 | 0.754 | 0.006 | 0.546 | 0.006 | 0.008 | <0.001 | 0.012 |
| rs1698041 | 0.955 | 0.011 | 0.700 | 0.013 | 0.015 | <0.001 | 0.040 |
| rs11713954 | 0.834 | 0.625 | 0.944 | 0.737 | 0.582 | 0.250 | 0.417 |
| rs2055426 | 0.481 | 0.003 | 0.290 | 0.005 | 0.003 | <0.001 | 0.033 |
| rs2937675 | 0.445 | 0.002 | 0.267 | 0.004 | 0.003 | <0.001 | 0.021 |
| rs1875518 | 0.676 | 0.005 | 0.435 | 0.010 | 0.007 | <0.001 | 0.034 |
| rs2937673 | 0.745 | 0.010 | 0.520 | 0.012 | 0.010 | <0.001 | 0.074 |
| rs1676232 | 0.342 | <0.001 | 0.171 | 0.001 | <0.001 | <0.001 | 0.037 |
| rs4855952 | 0.906 | 0.327 | 0.683 | 0.574 | 0.494 | 0.930 | 0.894 |
| rs1501874 | 0.563 | 0.022 | 0.828 | 0.094 | 0.059 | 0.100 | 0.337 |
| rs2937670 | 0.250 | 0.426 | 0.195 | 0.762 | 0.201 | 0.063 | 0.696 |
| rs9824498 | 0.522 | 0.719 | 0.549 | 0.745 | 0.874 | 0.951 | N/A |
| rs1979868 | 0.227 | 0.574 | 0.339 | 0.661 | 0.742 | 0.624 | 0.988 |
| rs1381801 | 0.110 | 0.822 | 0.192 | 0.675 | 0.763 | 0.765 | 0.565 |
| rs2937666 | 0.017 | 0.258 | 0.032 | 0.177 | 0.267 | 0.984 | 0.035 |
| rs1910044 | 0.596 | 0.604 | 0.670 | 0.581 | 0.567 | 0.776 | N/A |
| rs4855955 | 0.244 | 0.297 | 0.346 | 0.219 | 0.317 | 0.109 | 0.135 |
| rs6778437 | 0.071 | 0.856 | 0.098 | 0.877 | 0.610 | 0.979 | 0.989 |
| rs6795971 | 0.130 | 0.766 | 0.174 | 0.776 | 0.976 | 0.641 | 0.884 |
| rs1393192 | 0.167 | 0.493 | 0.258 | 0.368 | 0.915 | 0.576 | N/A |
| rs1466416 | 0.879 | 0.132 | 0.807 | 0.950 | 0.092 | 0.165 | 0.797 |

TABLE 6b
Association tests between case groups and controls in the full model adjusting for gender,
ethnicity, hypertension, body mass index, diabetes, dyslipidemia, and smoking history.

| | YA_aac | OA_aac | YA_aoo | OA_aoo | SA | LM | GC |
|---|---|---|---|---|---|---|---|
| SNP | (N = 301) | (N = 168) | (N = 358) | (N = 111) | (N = 202) | (N = 120) | (N = 420) |
| rs1513172 | 0.693 | 0.453 | 0.400 | 0.972 | 0.365 | 0.381 | 0.336 |
| rs6438389 | 0.131 | 0.148 | 0.137 | 0.076 | 0.158 | 0.344 | 0.176 |
| rs1513156 | 0.019 | 0.041 | 0.017 | 0.053 | 0.036 | 0.058 | 0.013 |
| rs11716267 | 0.348 | 0.300 | 0.835 | 0.798 | 0.506 | 0.264 | 0.519 |
| rs1398626 | 0.854 | 0.024 | 0.476 | 0.045 | 0.051 | 0.216 | 0.106 |
| rs1513162 | 0.436 | 0.702 | 0.683 | 0.899 | 0.938 | 0.735 | 0.334 |
| rs4075039 | 0.972 | 0.074 | 0.680 | 0.152 | 0.126 | 0.029 | 0.510 |
| rs7427839 | 0.443 | 0.732 | 0.539 | 0.353 | 0.444 | 0.347 | 0.720 |
| rs6790819 | 0.315 | 0.588 | 0.450 | 0.805 | 0.861 | 0.767 | 0.506 |
| rs4356827 | 0.413 | 0.176 | 0.362 | 0.091 | 0.145 | 0.171 | 0.116 |
| rs2927275 | 0.209 | 0.144 | 0.260 | 0.048 | 0.091 | 0.208 | 0.112 |
| rs1698042 | 0.383 | 0.014 | 0.233 | 0.016 | 0.017 | 0.023 | 0.430 |
| rs1501881 | 0.467 | 0.255 | 0.595 | 0.278 | 0.336 | 0.106 | 0.222 |
| rs1910040 | 0.816 | 0.142 | 0.952 | 0.219 | 0.190 | 0.136 | 0.081 |
| rs1501885 | 0.755 | 0.028 | 0.542 | 0.028 | 0.036 | 0.005 | N/A |
| rs1354152 | 0.768 | 0.041 | 0.571 | 0.037 | 0.053 | 0.007 | 0.080 |
| rs1698041 | 0.949 | 0.094 | 0.721 | 0.097 | 0.122 | 0.021 | 0.137 |
| rs11713954 | 0.430 | 0.689 | 0.617 | 0.602 | 0.618 | 0.819 | 0.878 |
| rs2055426 | 0.563 | 0.033 | 0.371 | 0.039 | 0.038 | 0.004 | 0.116 |
| rs2937675 | 0.503 | 0.027 | 0.329 | 0.029 | 0.031 | 0.004 | 0.087 |
| rs1875518 | 0.650 | 0.054 | 0.463 | 0.058 | 0.064 | 0.007 | 0.122 |
| rs2937673 | 0.800 | 0.075 | 0.615 | 0.061 | 0.082 | 0.010 | 0.244 |
| rs1676232 | 0.123 | 0.001 | 0.054 | 0.002 | 0.001 | <0.001 | 0.041 |
| rs4855952 | 0.614 | 0.347 | 0.404 | 0.550 | 0.528 | 0.998 | 0.724 |
| rs1501874 | 0.451 | 0.088 | 0.706 | 0.214 | 0.239 | 0.195 | 0.644 |
| rs2937670 | 0.109 | 0.178 | 0.096 | 0.337 | 0.077 | 0.013 | 0.906 |
| rs9824498 | 0.720 | 0.353 | 0.659 | 0.471 | 0.365 | 0.388 | N/A |
| rs1979868 | 0.187 | 0.723 | 0.221 | 0.518 | 0.856 | 0.719 | 0.235 |
| rs1381801 | 0.227 | 0.734 | 0.269 | 0.894 | 0.658 | 0.768 | 0.118 |
| rs2937666 | 0.080 | 0.171 | 0.110 | 0.184 | 0.262 | 0.961 | 0.083 |
| rs1910044 | 0.551 | 0.580 | 0.543 | 0.794 | 0.651 | 0.648 | N/A |
| rs4855955 | 0.550 | 0.154 | 0.640 | 0.073 | 0.148 | 0.037 | 0.552 |
| rs6778437 | 0.284 | 0.940 | 0.276 | 0.894 | 0.722 | 0.688 | 0.575 |
| rs6795971 | 0.429 | 0.712 | 0.431 | 0.588 | 0.925 | 0.411 | 0.601 |
| rs1393192 | 0.282 | 0.226 | 0.379 | 0.105 | 0.446 | 0.248 | N/A |
| rs1466416 | 0.542 | 0.205 | 0.683 | 0.951 | 0.163 | 0.238 | 0.588 |

Legend to Table 5b: YA_aac = young affected (CADi >23, age-at-catheterization <56); OA_aac = old affected (CADi >67, age-at-catheterization >=56); YA_aoo = young affected (CADi >23, age-of-onset <56); OA_aoo = old affected (CADi >67, age-of-onset >=56); SA = severe affected (CADi >67, regardless of age-of-onset); LM = left main affected (CADi ≧82, regardless of age-of-onset); GC = GENECARD probands. All the case groups were compared to CATHGEN controls (N = 204) using additive allele model. P-values < 0.05 are in bold. N/A, data was not available.

Since the original linkage was observed in families with early-onset CAD, our initial expectation was that any genetic association would be detected in the dataset with a younger AAC. Thus, it was surprising that the strongest associations were found in the OA_aac group. Realizing that AAC may not be a good surrogate for age-of-onset, subjects were subsequently reclassified on the basis of AOO to examine whether the association detected in the OA_aac was due to misclassification of individuals. Despite the fact that one third of OA_aac were reclassified into YA_AOO upon examination, the evidence for association remained in the OA_AOO group (Table 7), suggesting that the common feature driving the significant association in both the CATHGEN and GENECARD datasets is not related to age.

TABLE 7
Association analysis on SNPs immediately around rs1875518

| | Additive association test versus | | | | |
|---|---|---|---|---|---|
| | control, p-value* | | | | MAF |
| | YA_aoo | OA_aoo | SA | LM | control |
| SNP | (N = 358) | (N = 111) | (N = 202) | (N = 120) | (N = 204) |
| rs1698042 | 0.233 | 0.016 | 0.017 | 0.023 | 10% |
| rs1501881 | 0.595 | 0.278 | 0.336 | 0.106 | 42% |
| rs1910040 | 0.952 | 0.219 | 0.190 | 0.136 | 32% |
| rs1501885 | 0.542 | 0.028 | 0.036 | 0.005 | 49% |
| rs1354152 | 0.571 | 0.037 | 0.053 | 0.007 | 48% |
| rs1698041 | 0.721 | 0.097 | 0.122 | 0.021 | 49% |
| rs11713954 | 0.617 | 0.602 | 0.618 | 0.819 | 8% |
| rs2055426 | 0.371 | 0.039 | 0.038 | 0.004 | 48% |
| rs2937675 | 0.329 | 0.029 | 0.031 | 0.004 | 48% |
| rs1875518 | 0.463 | 0.058 | 0.064 | 0.007 | 50% |
| rs2937673 | 0.615 | 0.061 | 0.082 | 0.010 | 50% |
| rs1676232 | 0.054 | 0.002 | 0.001 | <0.001 | 44% |
| rs4855952 | 0.404 | 0.550 | 0.528 | 0.998 | 4% |
| rs1501874 | 0.706 | 0.214 | 0.239 | 0.195 | 10% |
| rs2937670 | 0.096 | 0.337 | 0.077 | 0.013 | 13% |
| rs9824498 | 0.659 | 0.471 | 0.365 | 0.388 | 23% |
| rs1979868 | 0.221 | 0.518 | 0.856 | 0.719 | 38% |
| rs1381801 | 0.269 | 0.894 | 0.658 | 0.768 | 42% |
| rs2937666 | 0.110 | 0.184 | 0.262 | 0.961 | 40% |

*A multivariable logistic regression model was used adjusting for gender, ethnicity, hypertension, diabetes mellitus, body mass index, dyslipidemia, and smoking history. P-values < 0.05 are in bold. YA_aoo = young affected (CADi >23, age-of-onset <56); OA_aoo = old affected (CADi >67, age-of-onset ≧56); SA = severe affected (CADi >67, regardless of age-of-onset); LM = left main affected (CADi ≧82, regardless of age-of-onset).

Therefore, we investigated the other major variable used in classifying the CATHGEN cases, CADi. Due to the higher threshold of CADi used to define the old affected, this group has more severe CAD when compared to the young affecteds (Table 4). The GENECARD probands also have a high burden of CAD, as evidenced by the high prevalence of previous coronary artery bypass grafting (CABG, 40.0%) and multiple-vessel CAD (47.1%). Hence, we constructed the severely affected set by including all subjects with CADi >67 (the CADi criteria used to define the old affected), regardless of age. The SA dataset (91 YA_AOO and 111 OA_AOO individuals) confirmed associations found in the OA_AOO group, suggesting that the associations are driven by the CADi but not AOO (Table 8). As higher CADi rankings are weighted by the presence of left main coronary artery disease (LMD) (Table 1), 60% of the SA subjects have LMD. Therefore, we composed a final subset, LM, of those individuals with LMD. Despite a smaller sample size, the associations became more significant in the LM than the SA group (Table 7), suggesting that the evidence for association was indeed driven by the individuals with LMD. The odds ratio for LMD risk after adjustment for the traditional CAD risk factors was 2.63 (95% CI: 1.43-4.83) for the risk allele of rs1676232 in the recessive model.

TABLE 8
Association analysis on SNPs immediately around rs1875518

| | Additive association test versus | | | | |
|---|---|---|---|---|---|
| | control, p-value* | | | | MAF |
| | YA_aoo | OA_aoo | SA | LM | control |
| SNP | (N = 358) | (N = 111) | (N = 202) | (N = 120) | (N = 204) |
| rs1698042 | 0.233 | 0.016 | 0.017 | 0.023 | 10% |
| rs1501881 | 0.595 | 0.278 | 0.336 | 0.106 | 42% |
| rs1910040 | 0.952 | 0.219 | 0.190 | 0.136 | 32% |
| rs1501885 | 0.542 | 0.028 | 0.036 | 0.005 | 49% |
| rs1354152 | 0.571 | 0.037 | 0.053 | 0.007 | 48% |
| rs1698041 | 0.721 | 0.097 | 0.122 | 0.021 | 49% |
| rs11713954 | 0.617 | 0.602 | 0.618 | 0.819 | 8% |
| rs2055426 | 0.371 | 0.039 | 0.038 | 0.004 | 48% |
| rs2937675 | 0.329 | 0.029 | 0.031 | 0.004 | 48% |
| rs1875518 | 0.463 | 0.058 | 0.064 | 0.007 | 50% |
| rs2937673 | 0.615 | 0.061 | 0.082 | 0.010 | 50% |
| rs1676232 | 0.054 | 0.002 | 0.001 | <0.001 | 44% |
| rs4855952 | 0.404 | 0.550 | 0.528 | 0.998 | 4% |
| rs1501874 | 0.706 | 0.214 | 0.239 | 0.195 | 10% |
| rs2937670 | 0.096 | 0.337 | 0.077 | 0.013 | 13% |
| rs9824498 | 0.659 | 0.471 | 0.365 | 0.388 | 23% |
| rs1979868 | 0.221 | 0.518 | 0.856 | 0.719 | 38% |
| rs1381801 | 0.269 | 0.894 | 0.658 | 0.768 | 42% |
| rs2937666 | 0.110 | 0.184 | 0.262 | 0.961 | 40% |

*A multivariable logistic regression model was used adjusting for gender, ethnicity, hypertension, diabetes mellitus, body mass index, dyslipidemia, and smoking history. P-values < 0.05 are in bold. YA_aoo = young affected (CADi >23, age-of-onset <56); OA_aoo = old affected (CADi >67, age-of-onset ≧56); SA = severe affected (CADi >67, regardless of age-of-onset); LM = left main affected (CADi ≧82, regardless of age-of-onset).

### Example 4

### Linkage Disequilibrium (LD)

LD relationships are shown in Table 9. Eight common (frequency >2%) haplotypes containing rs1676232 were estimated using haplotype tagging SNPs in moderate LD (r²>0.34) and accounted for 95% of all possible haplotypes in our sample (Table 9). Although this analysis slightly improved the association with GENECARD probands, overall it did not provide any additional information in the CATHGEN groups.

TABLE 9
Haplotype analysis using haplotype tagging SNPs

| Haplotype | | | | | Control | SA | | GC | |
|---|---|---|---|---|---|---|---|---|---|
| RS1676232 | RS2927275 | RS1501881 | RS1910040 | RS1875518 | Freq | Freq | p-value | Freq | p-value |
| A | A | C | A | A | 4% | 3% | 0.783 | 3% | 0.952 |
| A | A | C | A | G | 44% | 57% | 0.018 | 54% | 0.021 |
| A | G | C | A | G | 3% | 3% | 0.410 | 3% | 0.436 |
| G | A | C | A | A | 5% | 1% | 0.002 | 3% | 0.114 |
| G | A | T | A | A | 4% | 3% | 0.659 | 4% | 0.855 |
| G | A | T | G | A | 4% | 3% | 0.306 | 3% | 0.784 |
| G | G | T | A | A | 4% | 2% | 0.483 | 3% | 0.328 |
| G | G | T | G | A | 27% | 23% | 0.438 | 22% | 0.030 |

Haplotype frequency (Freq) was estimated using HaploStats in each group. Control = CATHGEN controls; SA = CATHGEN sever affected; GC = GENECARD probands. P-values < 0.05 are in bold.

### Example 5

### Identification of closest neighbor genes

The associated SNPs lie within an approximately 2.5 Mb region that does not harbor any annotated genes (http://www.ensembl.org, Human v27.35a.1). Distally, immunoglobin superfamily member 11 gene is about 1.4 Mb away, while proximally the 5′ end of the limbic system-associated membrane protein (LSAMP) gene resides approximately 1.1 Mb away. A recent report on the genomic structure of the mouse LSAMP gene identified an alternative exon 1 (exon 1a), located 1.6 Mb away from the originally described exon 1b (18).

As exon 1a had not yet been annotated to the current human genome assembly, we performed in silico analyses and found a similar gene structure lying 5′ to the publically annotated exon 1b of the human LSAMP gene. This positioned the associated SNPs within the unusually large alternative intron 1 of LSAMP between exon 1a and exon 1b.

### Example 6

### Expression of LSAMP

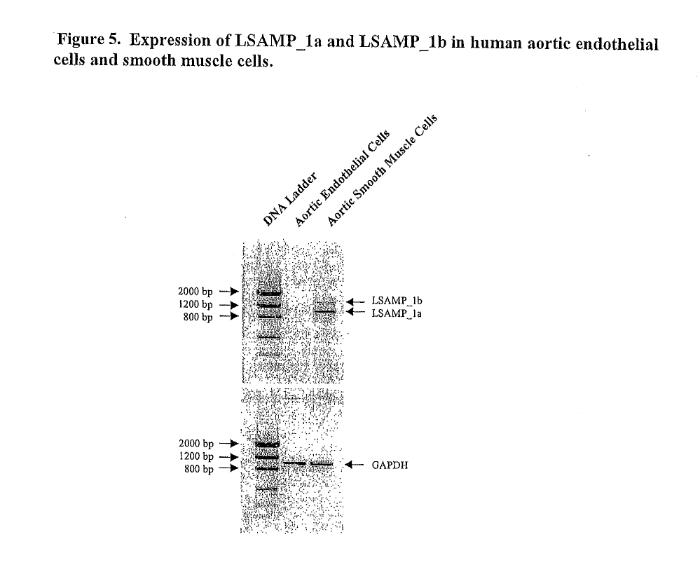

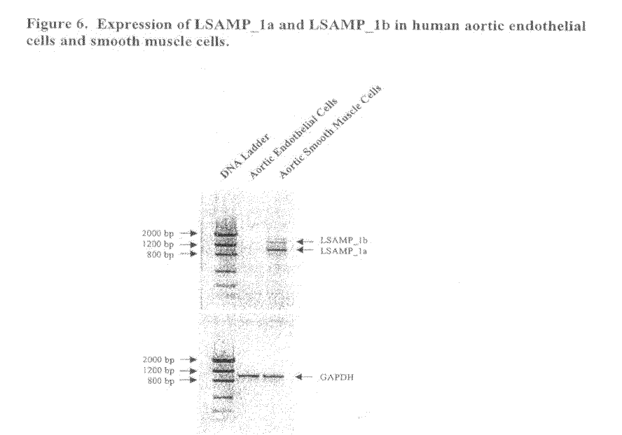

RT-PCR confirmed the existence of the alternative transcripts initiated by exon 1a (LSAMP_{—}1a) or exon 1b (LSAMP_{—}1b) in several human tissues (FIG. 5). Within human aorta, both LSAMP_{—}1a and LSAMP_{—}1b are expressed in the smooth muscle cells, where LSAMP_{—}1a was the predominant transcript, but none was expressed in the endothelial cells (FIG. 6).

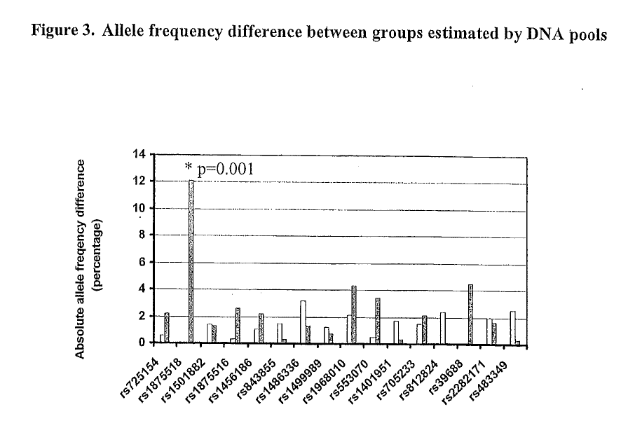

We examined the expression of LSAMP_{—}1a in 37 human aortas with varying degrees of atherosclerosis (15). The expression of LSAMP_{—}1a was decreased by 6.5 fold in aortas with severe atherosclerosis as compared to those with mild atherosclerosis (p<0.001, FIG. 3a).

Genotyping of the aortas for rs1676232 revealed that the CAD risk allele A was indeed associated with decreased LSAMP_{—}1a expression (p=0.05, FIG. 3b).

### REFERENCES

The disclosure of each reference cited is expressly incorporated herein.
(1) Shea S, Ottman R, Gabrieli C, Stein Z, Nichols A. Family history as an independent risk factor for coronary artery disease. J Am Coll Cardiol 1984; 4(4):793-801.
(2) Ten Kate L P, Boman H, Daiger S P, Motulsky A G. Familial aggregation of coronary heart disease and its relation to known genetic risk factors. Am J Cardiol 1982; 50(5):945-953.
(3) Harrap S B, Zammit K S, Wong Z Y, Williams F M, Bahlo M, Tonkin A M et al. Genome-wide linkage analysis of the acute coronary syndrome suggests a locus on chromosome 2. Arterioscler Thromb Vasc Biol 2002; 22(5):874-878.
(4) Broeckel U, Hengstenberg C, Mayer B, Holmer S, Martin L J, Comuzzie A G et al. A comprehensive linkage analysis for myocardial infarction and its related risk factors. Nat Genet. 2002; 30(2):210-214.
(5) Francke S, Manraj M, Lacquemant C, Lecoeur C, Lepretre F, Passa P et al. A genome-wide scan for coronary heart disease suggests in Indo-Mauritians a susceptibility locus on chromosome 16p13 and replicates linkage with the metabolic syndrome on 3q27. Hum Mol Genet. 2001; 10(24):2751-2765.
(6) Pajukanta P, Cargill M, Viitanen L, Nuotio I, Kareinen A, Perola M et al. Two loci on chromosomes 2 and X for premature coronary heart disease identified in early- and late-settlement populations of Finland. Am J Hum Genet. 2000; 67(6):1481-1493.
(7) Wang Q, Rao S, Shen G Q, Li L, Moliterno D J, Newby L K et al. Premature Myocardial Infarction Novel Susceptibility Locus on Chromosome 1P34-36 Identified by Genomewide Linkage Analysis. Am J Hum Genet. 2004; 74(2):262-271.
(8) Chiodini B D, Lewis C M. Meta-analysis of 4 coronary heart disease genome-wide linkage studies confirms a susceptibility locus on chromosome 3q. Arterioscler Thromb Vasc Biol 2003; 23(10):1863-1868.
(9) Hauser E R, Crossman D C, Granger C B, Haines J L, Jones C J, Mooser V et al. A genomewide scan for early-onset coronary artery disease in 438 families: the GENECARD Study. Am J Hum Genet. 2004; 75(3):436-447.
(10) Fortin D F, Califf R M, Pryor D B, Mark D B. The way of the future redux. Am J Cardiol 1995; 76(16):1177-1182.
(11) Hauser E R, Mooser V, Crossman D C, Haines J L, Jones C H, Winkelmann B R et al. Design of the genetics of early onset cardiovascular disease (GENECARD) study. Am Heart J 2003; 145(4):602-613.
(12) Smith L. R, Harrell F. E jr., Rankin J. S, Califf R. M, Pryor D. B, Muhlbaier L. H et al. Determinants of early versus late cardiac death in patients undergoing coronary artery bypass graft surgery. Circulation 84[5 Suppl], 111245-253. 1991. Ref Type: Abstract
(13) Kong D F, Shaw L K, Harrell F E, Muhlbaier L H, Lee K L, Califf R M et al. Predicting survival from the coronary arteriogram: an experience-based statistical index of coronary artery disease severity. Journal of the American College of Cardiology 39 (Suppl A), 327A. 2002. Ref Type: Abstract
(14) Hoogendoorn B, Owen M J, Oefner P J, Williams N, Austin J, O'Donovan M C. Genotyping single nucleotide polymorphisms by primer extension and high performance liquid chromatography. Hum Genet. 1999; 104:89-93.
(15) Seo D, Wang T, Dressman H, Herderick E E, Iversen E S, Dong C et al. Gene Expression Phenotypes of Atherosclerosis. Arterioscler Thromb Vasc Biol 2004; 24(10):1922-1927.
(16) Carlson C S, Eberle M A, Rieder M J, Yi Q, Kruglyak L, Nickerson D A. Selecting a maximally informative set of single-nucleotide polymorphisms for association analyses using linkage disequilibrium. Am J Hum Genet. 2004; 74(1):106-120.
(17) Abecasis G R, Cookson W O. GOLD—graphical overview of linkage disequilibrium. BioInformatics 2000; 16(2):182-183.
(18) Pimenta A F, Levitt P. Characterization of the genomic structure of the mouse limbic system-associated membrane protein (Lsamp) gene. Genomics 2004; 83(5):790-801.
(19) Fischer M, Broeckel U, Holmer S, Baessler A, Hengstenberg C, Mayer B et al. Distinct heritable patterns of angiographic coronary artery disease in families with myocardial infarction. Circulation 2005; 111(7):855-862.
(20) Khot U N, Khot M B, Bajzer C T, Sapp S K, Ohman E M, Brener S J et al. Prevalence of conventional risk factors in patients with coronary heart disease. JAMA 2003; 290(7):898-904.
(21) Eagle K A, Guyton R A, Davidoff R, Edwards F H, Ewy G A, Gardner T J et al. ACC/AHA 2004 guideline update for coronary artery bypass graft surgery: a report of the American College of Cardiology/American Heart Association Task Force on Practice Guidelines (Committee to Update the 1999 Guidelines for Coronary Artery Bypass Graft Surgery). Circulation 2004; 110 (14):e340-e437.
(22) Pimenta A F, Zhukareva V, Barbe M F, Reinoso B S, Grimley C, Henzel W et al. The limbic system-associated membrane protein is an Ig superfamily member that mediates selective neuronal growth and axon targeting. Neuron 1995; 15(2):287-297.
(23) McNamee C J, Reed J E, Howard M R, Lodge A P, Moss D J. Promotion of neuronal cell adhesion by members of the IgLON family occurs in the absence of either support or modification of neurite outgrowth. J Neurochem 2002; 80(6):941-948.
(24) Eagleson K L, Pimenta A F, Burns M M, Fairfull L D, Cornuet P K, Zhang L et al. Distinct domains of the limbic system-associated membrane protein (LAMP) mediate discrete effects on neurite outgrowth. Mol Cell Neurosci 2003; 24(3):725-740.
(25) Nelovkov A, Philips M A, Koks S, Vasar E. Rats with low exploratory activity in the elevated plus-maze have the increased expression of limbic system-associated membrane protein gene in the periaqueductal grey. Neurosci Lett 2003; 352(3):179-182.
(26) Chen J, Lui W O, Vos M D, Clark G J, Takahashi M, Schoumans J et al. The t(1; 3) breakpoint-spanning genes LSAMP and NORE1 are involved in clear cell renal cell carcinomas. Cancer Cell 2003; 4(5):405-413.
(27) Dermitzakis E T, Reymond A, Lyle R, Scamuffa N, Ucla C, Deutsch S et al. Numerous potentially functional but non-genic conserved sequences on human chromosome 21. Nature 2002; 420(6915):578-582.
(28) Hardison R C. Conserved noncoding sequences are reliable guides to regulatory elements. Trends Genet. 2000; 16(9):369-372.
(29) Kleinjan D J, van H, V. Position effect in human genetic disease. Hum Mol Genet. 1998; 7(10):1611-1618.
(30) de Kok Y J, Vossenaar E R, Cremers C W, Dahl N, Laporte J, Hu U et al. Identification of a hot spot for microdeletions in patients with X-linked deafness type 3 (DFN3) 900 kb proximal to the DFN3 gene POU3F4. Hum Mol Genet. 1996; 5(9):1229-1235.
(31) Kleinjan DA, van H, V. Long-range control of gene expression: emerging mechanisms and disruption in disease. Am J Hum Genet. 2005; 76(1):8-32.
(32) Lettice L A, Horikoshi T, Heaney S J, van Baren M J, van der Linde H C, Breedveld G J et al. Disruption of a long-range cis-acting regulator for Shh causes preaxial polydactyl). Proc Natl Acad Sci USA 2002; 99(11):7548-7553.
(33) Nobrega M A, Ovcharenko I, Afzal V, Rubin E M. Scanning human gene deserts for long-range enhancers. Science 2003; 302(5644):413.
(34) Dong C, Yoon W, Goldschmidt-Clermont P J. DNA methylation and atherosclerosis. J Nutr 2002; 132 (8 Suppl):2406S-2409S.
(35) Li Y J, Scott W K, Hedges D J, Zhang F, Gaskell P C, Nance M A et al. Age at onset in two common neurodegenerative diseases is genetically controlled. Am J Hum Genet. 2002; 70(4):985-993.
(36) Li Y J, Hauser M A, Scott W K, Martin E R, Booze M W, Qin X J et al. Apolipoprotein E controls the risk and age at onset of Parkinson Disease. Neurology 2004; 62(11):2005-2009.
(37) Stenger J E, Xu H, Haynes C, Hauser E R, Pericak-Vance M A, Goldschmidt-Clermont P J et al. Statistical Viewer: a tool to upload and integrate linkage and association data as plots displayed within the Ensembl genome browser. BMC Bioinformatics 2005; 6(1):95.
(38) Dausset J, Cann H, Cohen D, Lathrop M, Lalouel J M, White R. Centre d′ etude du polymorphisme humanin (CEPH): collaborative genetic mapping of the human genome. Genomics 1990; 6:575-577.

We claim:

1. A method of identifying a human subject having an increased risk of developing cardiovascular disease, comprising determining an expression level of exon 1a of LSAMP in a human cardiovascular tissue sample, wherein a decrease in the expression level of exon 1a of LSAMP as compared to a control identifies the subject as having an increased risk of developing cardiovascular disease.

2. The method of claim 1, wherein the determined expression level of exon 1a of LSAMP is normalized to gene expression of a gene whose expression is deemed substantially constant in cardiovascular tissues.

3. The method of claim 1, wherein the determined expression level of exon 1a of LSAMP is normalized to gene expression of a glyceraldehyde phosphate dehydrogenase gene.

4. The method of claim 1, wherein expression of LSAMP exon 1a mRNA is determined.

5. The method of claim 1, wherein the human cardiovascular tissue sample is from an aorta.

6. The method of claim 4, wherein reverse transcription-polymerase chain reaction (RT-PCR) is employed to determine expression of mRNA.

7. The method of claim 1, wherein expression of LSAMP protein is determined.

8. The method of claim 1, wherein the cardiovascular disease is coronary artery disease.

9. The method of claim 1, wherein the cardiovascular disease is arteriosclerosis.

10. The method of claim 1, wherein the cardiovascular disease is left main disease.

11-68. (canceled)

69. The method of claim 1, further comprising the steps of determining a factor selected from the group consisting of level of triglycerides, levels of cholesterol, diabetes mellitus, hypertension, family history, and cigarette smoking, and using said determination in combination with determination of the expression level of exon 1a in identifying increased risk of developing cardiovascular disease.

70. The method of claim 1, further comprising the steps of performing a test selected from the group consisting of an echocardiogram, a stress test, a blood pressure measurement, and an ejection fraction measure, and using the results of the test in combination with the determination of the expression level of exon 1a in identifying increased risk of developing cardiovascular disease.

71-96. (canceled)
